(19) United States
(12) Patent Application Publication
(10) Pub. No.: US 2026/0157762 A1
(43) Pub. Date: Jun. 11, 2026

(54) Glenoid Drill Guide

(71) Applicant: Stryker Corporation, Portage, MI (US)

(72) Inventors: Jeremy Graul, Denver, CO (US); Jonas R. Rudzki, Bethesda, MD (US); Kyle Craig Pilgeram, San Jose, CA (US)

(73) Assignee: Stryker Corporation, Portage, MI (US)

(21) Appl. No.: 19/410,524

(22) Filed: Dec. 5, 2025

Related U.S. Application Data
(60) Provisional application No. 63/730,724, filed on Dec. 11, 2024.

(51) Int. Cl. A61B 17/17; A61B 17/00; A61B 17/84

(52) U.S. Cl. CPC A61B 17/1778; A61B 17/1796; A61B 17/848; A61B 2017/00477

(57) ABSTRACT

Glenoid drill guides and methods for repairing glenoid bone loss use a guide body defining first and second lumens. An implant insert is configured to be removably secured in the first lumen. A first bone block engagement insert defining a lumen is configured to be removably secured in a lumen of the implant insert when secured in the guide body such that the distal end of the first bone block engagement insert is distal to the guide body distal end. A second bone block engagement insert defining a lumen is configured to be removably secured in the second lumen such that the distal end of the second bone block engagement insert is distal to the guide body distal end. The distal ends of the first and second bone block engagement inserts are configured to engage a bone block to be secured to a glenoid bone.

### CROSS-REFERENCE TO RELATED APPLICATIONS

This application claims priority to U.S. Provisional Patent Application No. 63/730,724, titled “Glenoid Drill Guide,” filed on Dec. 11, 2024. The entire disclosure of the provisional application is hereby incorporated by reference in its entirety.

### BACKGROUND

Shoulder instability is a condition in which the head of the humerus (the ball portion of the shoulder joint) slips out of the glenoid, causing pain, discomfort, and reduced mobility. One of the common causes of shoulder instability is glenoid bone loss, which occurs when a portion of the glenoid rim is fractured or worn away due to trauma, disease, or degeneration. Glenoid bone loss reduces the depth and width of the glenoid socket, making it easier for the humeral head to dislocate.

One of the surgical procedures for treating glenoid bone loss is the Latarjet procedure, which involves transferring a segment of bone from the coracoid process (a bony projection on the scapula) to the anterior-inferior region of the glenoid. The transferred bone segment, also known as a bone block or graft, acts as a buttress to prevent the humeral head from escaping the glenoid socket. The bone block also provides a source of living tissue that can promote healing and integration with the glenoid.

However, the success of the Latarjet procedure often depends on the accurate positioning and alignment of the bone block relative to the glenoid. According to some aspects, is may be desired to align the bone block with the articular surface of the glenoid. The articular surface of the glenoid is the smooth surface formed by cartilage covering the bone of the glenoid and that contacts the humeral head. According to some other aspects, it may be desired to align the bone block with the surface of the glenoid bone underlying the articular surface. A typical goal of the bone block placement is to match the surface geometry of the glenoid, or more particularly the glenoid articular surface or underlying surface of the glenoid bone, to enable normal range of motion and stability of the shoulder joint. For instance, it is typically desired for the placed bone block to be flush with the glenoid articular surface or underlying glenoid bone surface, to be neither too medial nor too lateral relative to the glenoid, and have a transverse screw angle according to the surgeon's desired position, which may be parallel or nearly parallel to the glenoid articular surface or underlying bone surface of the glenoid.

Existing methods and devices for guiding the placement and orientation of the bone block are often inadequate, imprecise, or cumbersome. For example, some methods rely on freehand drilling or manual measurement, which are prone to human error and variability. Some devices use fixed or predetermined angles or dimensions, which may not suit the individual anatomy and pathology of each patient. Some devices are complex, bulky, or difficult to use, requiring multiple steps, adjustments, or components.

Therefore, there is a need for a glenoid drill guide that can overcome or eliminate the aforementioned challenges or problems, and provide a simple, reliable, and effective way of repairing glenoid bone loss with a bone block or graft.

### SUMMARY

In one exemplary aspect, a glenoid drill guide includes a guide body having proximal and distal ends and defining first and second lumens each extending between the proximal and distal ends. The glenoid drill guide also includes first and second bone block engagement inserts each having proximal and distal ends. The first bone block engagement insert defines a first lumen extending between the proximal and distal ends of the first bone block engagement insert, and the second bone block engagement insert defines a second lumen extending between the proximal and distal ends of the second bone block engagement insert. The distal ends of the first and second bone block engagement inserts are configured to engage a bone block to be secured to a glenoid bone. The glenoid drill guide also includes an implant insert having proximal and distal ends and defining a lumen extending between the proximal and distal ends of the implant insert. The implant insert is configured to be removably secured in the first lumen of the guide body such that the distal end of the implant insert is proximal to or flush with the distal end of the guide body. The first bone block engagement insert is configured to be removably secured in the lumen of the implant insert when the implant insert is secured in the first lumen of the guide body such that the distal end of the first bone block engagement insert is distal to the distal end of the guide body. The second bone block engagement insert is configured to be removably secured in the second lumen of the guide body such that the distal end of the second bone block engagement insert is distal to the distal end of the guide body.

In another exemplary aspect, a glenoid drill guide includes a guide body having proximal and distal ends and defining a lumen extending between the proximal and distal ends. The glenoid drill guide also includes a bone block engagement portion disposed at the distal end of the guide body and configured to engage a bone block to be secured to a glenoid bone. The glenoid drill guide also includes an outrigger arm including a proximal portion and a distal portion. The proximal portion is secured to the guide body, and the distal portion extends from the proximal portion to beyond the distal end of the guide body. The distal portion of the outrigger arm is configured in combination with the bone block engagement portion to retain the bone block at the distal end of the guide body. The outrigger arm is also adjustable between a first distance from the bone block engagement portion and a second distance from the bone block engagement portion, the first distance being greater than the second distance.

In another exemplary aspect, a method of repairing glenoid bone loss includes providing a guide body having proximal and distal ends and defining first and second lumens each extending between the proximal and distal ends. The method also includes securing an implant insert in the first lumen of the guide body such that a distal end of the implant insert is proximal to or flush with the distal end of the guide body. The implant insert defines a lumen extending between a proximal end and the distal end of the implant insert. The method also includes securing a first bone block engagement insert in the lumen of the secured implant insert such that a distal end of the first bone block engagement insert is distal to the distal end of the guide body, where the first bone block engagement insert defines a first lumen extending between a proximal end and the distal end of the first bone block engagement insert. The method also includes securing a second bone block engagement insert in the second lumen of the guide body such that a distal end of the second bone block engagement insert is distal to the distal end of the guide body, where the second bone block engagement insert defines a second lumen extending between a proximal end and the distal end of the second bone block engagement insert. The method also includes coupling a bone block to the distal ends of the secured first and second bone block engagement inserts; maneuvering the guide body with the coupled bone block to position the bone block against a glenoid bone; and inserting a k-wire through the first lumen of the secured first bone block engagement insert or the second lumen of the secured second bone block engagement insert while the coupled bone block is positioned against the glenoid bone to pin the bone block to the glenoid bone. The method also includes removing the first bone block engagement insert from the lumen of the secured implant insert after the k-wire is inserted; drilling into the bone block through the lumen of the secured implant insert after the first bone block engagement insert is removed, and removing the implant insert from the first lumen of the guide body after the bone block is drilled. The method also includes passing an implant through the first lumen of the guide body to secure the bone block to the glenoid bone after the implant insert is removed.

In another exemplary aspect, method of repairing glenoid bone loss includes providing a glenoid drill guide, the glenoid drill guide including a guide body, a bone block engagement portion disposed at a distal end of the guide body, and an outrigger arm. The guide body defines a lumen extending between a proximal end and the distal end of the guide body, and the outrigger arm includes a proximal portion secured to the guide body and a distal portion extending from the proximal portion to beyond the distal end of the guide body. The method also includes coupling a bone block to the bone block engagement portion; adjusting a position the outrigger arm relative to the guide body such that the outrigger arm engages the coupled bone block; and maneuvering the glenoid drill guide with the engaged bone block to position the bone block against a glenoid bone. The method also includes inserting a k-wire through the lumen of the guide body while the engaged bone block is positioned against the glenoid bone to pin the bone block to the glenoid bone, and securing the bone block to the glenoid bone with an implant while the k-wire holds the bone block in place against the glenoid bone.

In another exemplary aspect, a method of repairing glenoid bone loss includes providing a guide body having proximal and distal ends and defining a lumen extending between the proximal and distal ends. The method also includes securing a bone block engagement insert in the lumen of the guide body such that a distal end of the bone block engagement insert is distal to the distal end of the guide body, where the bone block engagement insert defines a lumen extending between a proximal end and the distal end of the bone block engagement insert. The method also includes coupling a bone block to the distal end of the secured bone block engagement insert; maneuvering the guide body with the coupled bone block to position the bone block against a glenoid bone; and inserting a k-wire through the lumen of the secured bone block engagement insert while the coupled bone block is positioned against the glenoid bone to pin the bone block to the glenoid bone. The method also includes removing the bone block engagement insert from the lumen of the guide body and over the inserted k-wire; drilling into the bone block through the lumen of the guide body and over the inserted k-wire after the bone block engagement insert is removed; and securing the bone block to the glenoid bone with an implant after the bone block is drilled.

Other objects, features and advantages of the present invention will be readily appreciated as the same becomes better understood after reading the subsequent description taken in connection with the accompanying drawings.

### BRIEF DESCRIPTION OF THE DRAWINGS

Advantages will be readily appreciated as the same becomes better understood by reference to the following detailed description when considered in connection with the accompanying drawings, wherein:

FIG. 1 is a perspective view of a glenoid drill guide for repairing glenoid bone loss according to an exemplary aspect.

FIG. 2 is an exploded perspective view of the glenoid drill guide of FIG. 1 according to an exemplary aspect.

FIG. 3 is a perspective view of an implant insert of the glenoid drill guide of FIG. 1 according to an exemplary aspect.

FIG. 4 is a perspective view of a guide body of the glenoid drill guide of FIG. 1 according to an exemplary aspect.

FIGS. 5A-5C are perspective views of the implant insert of FIG. 3 being secured in the guide body of FIG. 4 according to an exemplary aspect.

FIG. 6 is a cross-sectional view of the glenoid drill guide of FIG. 1 taken along a longitudinal axis of a lumen of the glenoid drill guide according to an exemplary aspect.

FIG. 7 is a perspective view of a bone block engagement insert of the glenoid drill guide of FIG. 1 according to an exemplary aspect.

FIG. 8 is a cross-sectional view of the glenoid drill guide of FIG. 1 taken along a lateral axis of the glenoid drill guide according to an exemplary aspect.

FIGS. 9A and 9B are additional perspective views of the glenoid drill guide of FIG. 1 according to an exemplary aspect.

FIG. 10 is a flowchart of a method for repairing glenoid bone loss using the glenoid drill guide of FIG. 1 according to an exemplary aspect.

FIGS. 11A and 11B are side perspective views of a bone block being coupled to the glenoid drill guide of FIG. 1 according to an exemplary aspect.

FIG. 12 is a perspective view of the glenoid drill guide of FIG. 1 having been maneuvered to position the coupled bone block against a glenoid bone according to an exemplary aspect.

FIG. 13 is a perspective view of the glenoid drill guide of FIG. 1 as maneuvered in FIG. 12 with a first K-wire having been inserted through a first lumen of the glenoid drill guide and a second K-wire having been inserted through a second lumen of the glenoid drill guide to pin the bone block to the glenoid bone according to an exemplary aspect.

FIG. 14 is a perspective view of the glenoid drill guide of FIG. 1 as maneuvered in FIG. 12 with first pilot holes having been formed in the bone block and glenoid bone by a drill inserted through the first lumen of the glenoid drill guide over the inserted first K-wire according to an exemplary aspect.

FIG. 15 is a perspective view of a first implant being passed over the inserted first K-wire, through the first lumen of the glenoid drill guide of FIG. 1 as maneuvered in FIG. 12, and into the first pilot holes formed in the bone block and glenoid bone to secure the bone block to the glenoid bone according to an exemplary aspect.

FIG. 16 is a perspective view of the bone block having been secured to the glenoid bone with the first implant of FIG. 15, with the glenoid drill guide having been removed from the bone block, and with a second implant being inserted into second pilot holes formed in the bone block and glenoid bone over the inserted second K-wire to further secure the bone block to the glenoid bone according to an exemplary aspect.

### DETAILED DESCRIPTION

The present disclosure generally relates to glenoid drill guides and related methods for repairing glenoid bone loss, such as that caused by shoulder instability, arthritis, or trauma. The glenoid drill guides are designed to facilitate the placement of a bone block onto the glenoid bone and secure it with one or more implants. The bone block can be harvested from the patient's own body or obtained from a donor. The bone block can restore the glenoid rim and provide stability and congruency to the glenohumeral joint.

A glenoid drill guide as described herein may be a modular device that is configured to assist a practitioner in performance of different steps of a glenoid repair procedure. To this end, the glenoid drill guide may include a guide body defining one or more lumens. Each lumen may be sized and/or shaped to assist a different step of the procedure, and may also be configured to receive at least one insert that adapts the lumen to assist performance of a different step of the procedure. Such procedure steps may include one or more of positioning a bone block against a glenoid bone to be repaired, inserting one or more K-wires into the bone block and glenoid bone to pin the bone block in place against the glenoid bone, forming one or more pilot holes in the bone block and glenoid bone, and securing the bone block to the glenoid bone with one or more implants.

In some implementations, the glenoid drill guide may include one or more interfaces for selective attachment of a handle and/or an outrigger arm to the guide body. The handle may provide a convenient grip for the practitioner to manipulate the glenoid drill guide, and the outrigger arm may help retain the bone block against a distal end of the guide body as the guide body is maneuvered to position of the bone block against the glenoid bone. The outrigger arm may also help ensure proper alignment of the bone block with the glenoid bone. In some examples, the guide body may include multiple handle mounting interfaces for attaching the handle in different orientations relative to the guide body, and/or may include multiple arm mounting interfaces for attaching the outrigger arm in different orientations relative to the guide body. Such interfaces enable customization of the glenoid drill guide to support treating varying patient anatomies and/or varying practitioner preferences. These and other advantages of the present disclosure are described in more detail below.

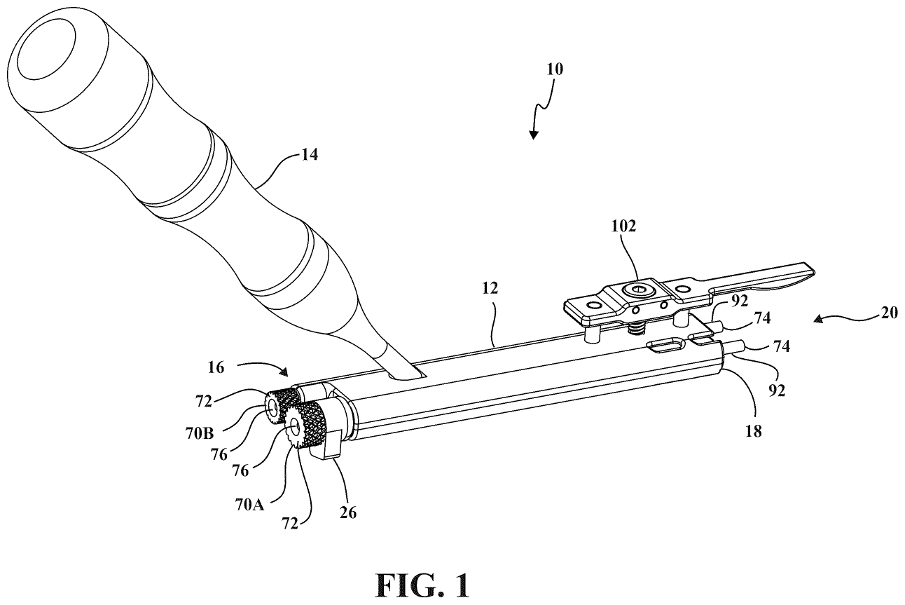

FIG. 1 illustrates a glenoid drill guide 10 in accordance with the present disclosure. As shown in the illustrated example, the glenoid drill guide 10 may include a guide body 12 and a handle 14 coupled to the guide body 12. The guide body 12 may have an elongated length extending between a proximal end 16 and a distal end 18 of the guide body 12. As used herein, “proximal” may be understood to mean towards the practitioner holding the glenoid drill guide 10, away from the surgical site to which the glenoid drill guide 10 is applied, and “distal” may be understood to mean away from the practitioner, towards the surgical site to which the glenoid drill guide 10 is applied.

The handle 14 may be coupled to the guide body 12 between the proximal end 16 and distal end 18, and may extend outwardly from the guide body 12 to define an interface for a practitioner to grip and maneuver the glenoid drill guide 10 relative to a glenoid bone being treated. In some implementations, the handle 14 may extend both outwardly from the guide body 12 and towards the proximal end 16 of the guide body 12 such that the handle 14 forms an acute angle with the guide body 12. Additionally or alternatively, and as described in more detail below, the handle 14 may be removably coupleable to the guide body 12 in different orientations relative to the guide body 12 to support treatment of different patient anatomies and/or different practitioner preferences.

The glenoid drill guide 10 may further include a bone block engagement portion 20 disposed at the distal end 18 of the guide body 12. During a surgical procedure such as a Latarjet procedure, a bone block may be removably coupled to the bone block engagement portion 20 of the glenoid drill guide 10. The bone block engagement portion 20 may be configured to retain the coupled bone block against the distal end 18 of the guide body 12 while a practitioner maneuvers the glenoid drill guide 10 using the handle 14 to position the coupled bone block against a glenoid bone to be treated. The practitioner may then proceed to secure the bone block to the glenoid bone while using the handle 14 to hold the bone block against the glenoid bone in a desired position, as described in more detail below.

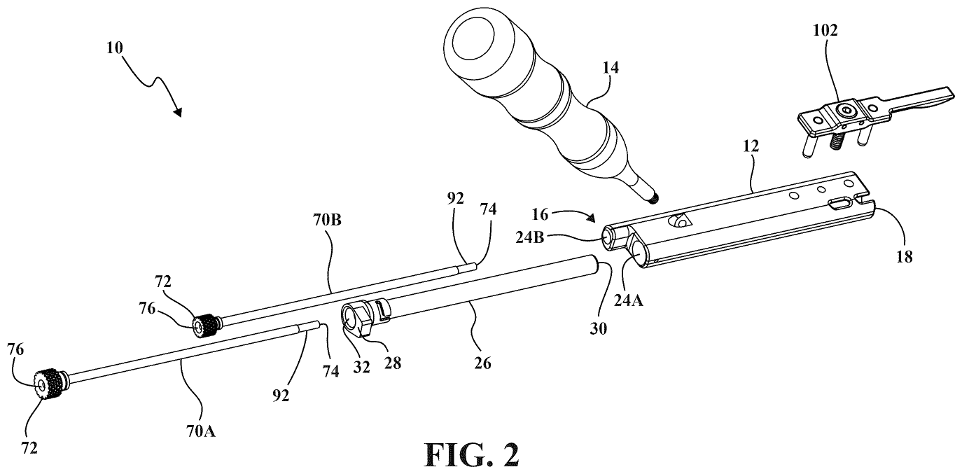

FIG. 2 illustrates an exploded view of the glenoid drill guide 10 of FIG. 1. As shown in the illustrated example, the guide body 12 of the glenoid drill guide 10 may define one or more lumens 24 each extending between the proximal end 16 and the distal end 18 of the guide body 12. In the illustrated example, the guide body 12 defines two such lumens 24, namely a first lumen 24A and a second lumen 24B. Each lumen 24 of the guide body 12 may define a longitudinal axis extending between the proximal end 16 and distal end 18 of the guide body 12, and may be open at the proximal end 16 and distal end 18 of the guide body 12 to allow insertion of a surgical instrument and/or implant through the lumen 24 and into a bone block coupled to the bone block engagement portion 20 and a glenoid bone against which the coupled bone block has been positioned. Each lumen 24 may include a transverse cross-section that is perpendicular to and generally uniform along the longitudinal axis the lumen 24. To guide an inserted surgical instrument and/or implant into a coupled bone block and glenoid bone according to a desired pose, the transverse cross-section of each lumen 24 may be sized to enable longitudinal movement of the surgical instrument and/or implant through the lumen 24 while limiting transverse movement of the inserted surgical instrument and/or implant within the lumen 24.

In some implementations, the transverse cross-section of each lumen 24 may differ in size and/or shape from that of each other lumen 24 so as to accommodate and guide different types of instruments and/or implants, such as to facilitate different steps of the procedure. For instance, the first lumen 24A and not the second lumen 24B may have a transverse cross-section that is sized and/or shaped to accommodate and guide insertion of an implant into a coupled bone block and glenoid bone. The second lumen 24B may have a transverse cross-section that is conversely sized and/or shaped to accommodate and guide insertion of a drill bit into a coupled bone block and glenoid bone to form a pilot hole for an implant.

In some implementations, the guide body 12 may define only one lumen 24, such as the first lumen 24A, able to accommodate and guide insertion of an implant into a coupled bone block and glenoid bone. Each other defined lumen 24, such as one or more of the second lumens 24B, may have a smaller transverse cross-section that is able to accommodate and guide insertion of a drill bit and/or K-wire into the coupled bone block and glenoid bone, but not able to accommodate an implant like the first lumen 24A. This configuration of the defined lumens 24 enables a reduction in the overall dimensions of the glenoid drill guide 10 compared to a drill guide defining a same number of lumens 24 and including multiple first lumens 24A able to accommodate an implant. Such reduction in size is especially beneficial in a surgical environment in which space and/or visibility may already be limited.

In some implementations, the glenoid drill guide 10 may also include one or more inserts configured to be releasably secured in the lumens 24 of the guide body 12. Each insert releasably secured in a given lumen 24 may be configured to adapt the lumen 24 to accommodate and guide an alternative instrument and/or implant into the bone block and glenoid bone.

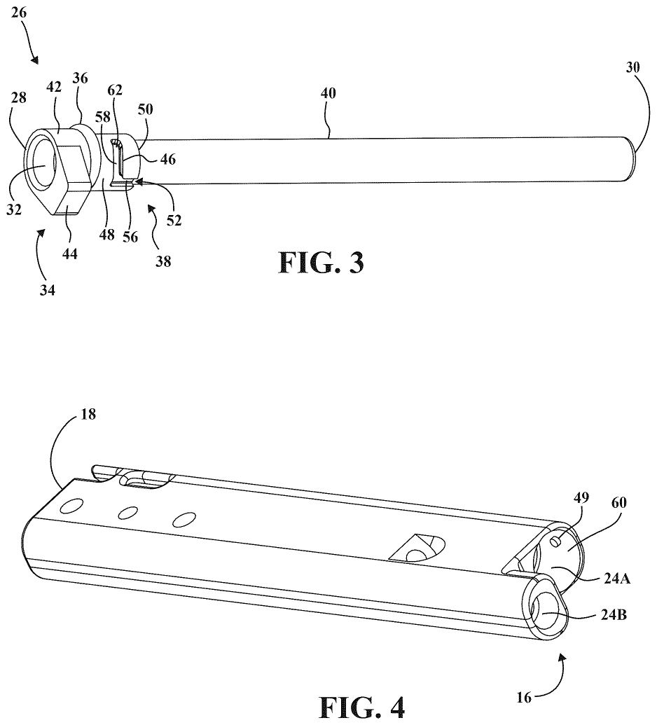

As one example, FIG. 3 illustrates an implant insert 26 that may be configured to be releasably secured in the first lumen 24A of the guide body 12. The implant insert 26 may be configured to adapt the first lumen 24A to accommodate and guide a drill bit into a coupled bone block and glenoid bone. To this end, the implant insert 26 may have a proximal end 28 and a distal end 30, and may define a lumen 32 extending between the proximal end 28 and distal end 30. The lumen 32 may define a longitudinal axis extending between the proximal end 28 and the distal end 30, and may be open at the proximal end 28 and distal end 30 to allow insertion of a drill bit through the lumen 32 and into a coupled bone block and glenoid bone when the implant insert 26 is releasably secured in the first lumen 24A of the guide body 12. The lumen 32 of the implant insert 26 may have a transverse cross-section that is perpendicular to and generally uniform along the longitudinal axis of the lumen 32. Similar to the second lumen 24B of the guide body 12, the transverse cross-section of the lumen 32 of the implant insert 26 may be sized and/or shaped for enabling longitudinal movement of a drill bit through the lumen 32 while limiting transverse movement of the drill bit within the lumen 32.

The implant insert 26 may include a handle 34, a compression member 36, a coupler 38, and a stem 40. The handle 34 may be disposed at the proximal end 28 of the implant insert 26, and may define an interface for being contacted by a practitioner to facilitate movement of the implant insert 26 relative to the guide body 12, as described in more detail below. In the illustrated example, the handle 34 is realized as an enlarged head 42 with a finger tab 44 protruding radially therefrom.

The coupler 38 may be positioned distal to the handle 34, and may be configured to releasably secure the implant insert 26 in the first lumen 24A. When the implant insert 26 is fully seated in the first lumen 24A, the coupler 38 may be moveable between a first position relative to the guide body 12 in which the implant insert 26 is able to be removed from the first lumen 24A in a proximal direction, and a second position relative to the guide body 12 in which longitudinal movement of the implant insert 26 relative to the first lumen 24A is prevented. When the coupler 38 is in the second position, other types of inserts may be releasably secured in and removed from the lumen 32 of the implant insert 26 without also removing or dislodging the implant insert 26 from the first lumen 24A. In some implementations, the coupler 38 may be moveable between the first and second positions by rotating the implant insert 26 relative to the guide body 12 when fully seated in the first lumen 24A, as described in more detail below.

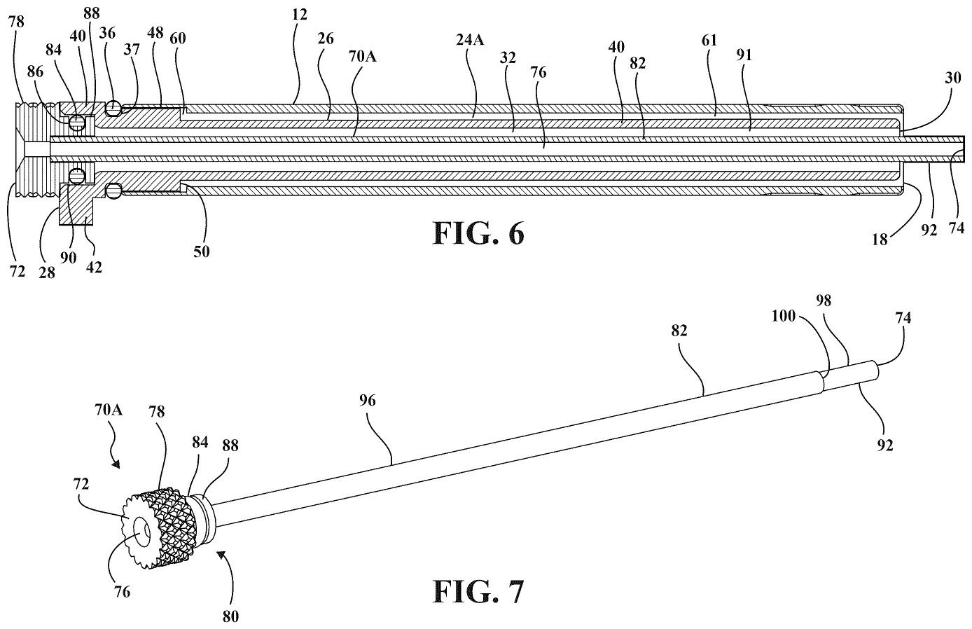

The compression member 36, which may be realized as an O-ring disposed in a groove 37 (FIG. 6) formed in the implant insert 26, may be positioned between the handle 34 and coupler 38. The stem 40 may extend distally from the coupler 38 and terminate at the distal end 30 of the implant insert 26.

FIG. 4 illustrates another view of the guide body 12 of the glenoid drill guide 10. Referring to FIGS. 3 and 4, in some implementations, the coupler 38 may be realized as a J-lock connector including a J-shaped channel 46 defined in an outer surface 48 of the implant insert 26. The J-shaped channel 46 may be configured to cooperate with a pin 49 disposed in the first lumen 24A adjacent the proximal end 16 of the guide body 12 to prevent longitudinal movement of the implant insert 26 relative to the first lumen 24A when the implant insert 26 is in the second position. More specifically, the outer surface 48 of the implant insert 26 may be spaced radially outward from the stem 40 so as to form a shoulder 50 that transversely extends between the stem 40 and the outer surface 48. The shoulder 50 may define an opening 52 configured to provide entry for the pin 49 into the J-shaped channel 46 as the implant insert 26 is inserted into the first lumen 24A. The J-shaped channel 46 may include a longitudinal channel portion 56 with one end defining the opening 52 and the other end connected to a transverse channel portion 58 of the J-shaped channel 46.

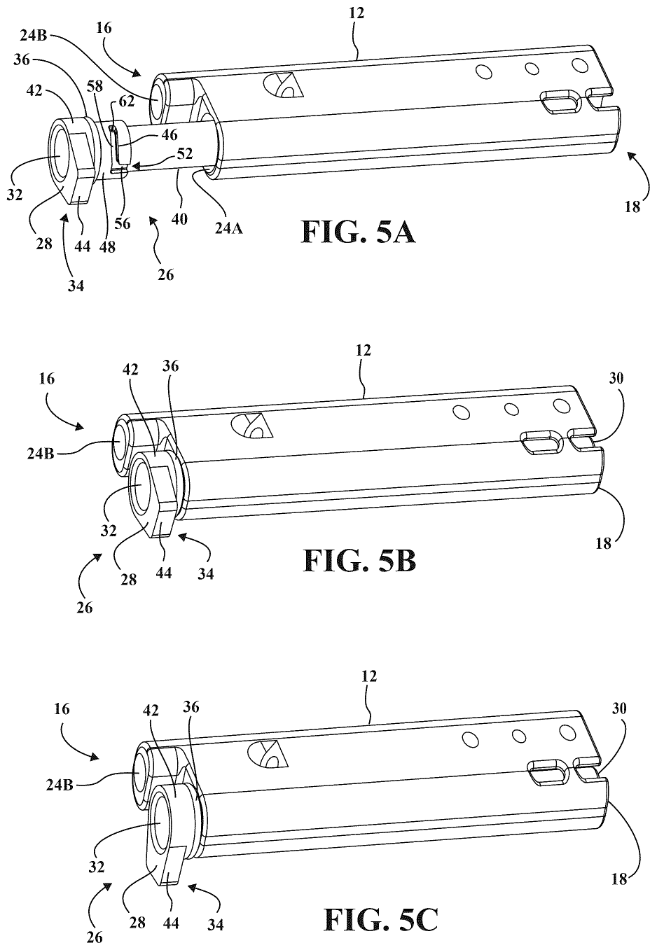

FIGS. 5A-5C illustrate the implant insert 26 being releasably secured in the first lumen 24A, and FIG. 6 illustrates an exemplary cross-section of the glenoid drill guide 10 taken along the longitudinal axis of the first lumen 24A. As illustrated in FIG. 5A, the implant insert 26 may be releasably secured in the first lumen 24A of the guide body 12 by first inserting the distal end 30 of the implant insert 26 into the first lumen 24A from the proximal end 16 of the guide body 12. As the implant insert 26 is further inserted into the first lumen 24A such as illustrated in FIG. 5B, the pin 49 may enter the longitudinal channel portion 56 of the J-shaped channel 46 via the opening 52.

As illustrated in FIG. 6, in some implementations, the first lumen 24A may include an enlarged proximal portion 60 for receiving the coupler 38 of the implant insert 26 when fully seated in the first lumen 24A, and a distal portion 61 for receiving the stem 40 of the implant insert 26 when fully seated in the first lumen 24A. As illustrated in FIG. 4, the pin 49 may extend radially inward into the enlarged proximal portion 60 of the first lumen 24A, and may be configured enter the longitudinal channel portion 56 of the J-shaped channel 46 via the opening 52 as the coupler 38 is received in the enlarged proximal portion 60 of the first lumen 24A. The pin 49 being disposed in the longitudinal channel portion 56 of the J-shaped channel 46 may correspond to the first position of the coupler 38 in which the implant insert 26 is able to be removed from the first lumen 24A as described above.

Once the pin 49 has entered the longitudinal channel portion 56 of the J-shaped channel 46, further force may be applied by the practitioner against the handle 34 of the implant insert 26 in the distal direction to compress the compression member 36 between the enlarged head 42 of the handle 34 and the proximal end 16 of the guide body 12, which may cause the pin 49 to move further into the longitudinal channel portion 56 of the J-shaped channel 46 and into alignment with the transverse channel portion 58 of the J-shaped channel 46. While the compression member 36 is in such compressed state, the implant insert 26 may be rotated relative to the first lumen 24A, such as via the finger tab 44. Rotation of the implant insert 26 may cause the transverse channel portion 58 to move with respect to the pin 49 until a closed end portion 62 of the transverse channel portion 58 that is opposite the longitudinal channel portion 56 abuts the pin 49. At this point, interference between the pin 49 and the closed end portion 62 of the transverse channel portion 58 may function to prevent the implant insert 26 from being removed from the first lumen 24A without first moving the coupler 38 to the first position. The pin 49 being disposed at the closed end portion 62 of the transverse channel portion 58 may thus correspond to the second position of the coupler 38 in which the implant insert 26 is prevented from being removed from the first lumen 24A as described above.

FIG. 5C illustrates the implant insert 26 having been inserted in the first lumen 24A and rotated to the second position so as to be releasably secured in the first lumen 24A. As shown in the illustrated examples of FIGS. 5C and 6, the implant insert 26 may have an elongate length extending between its proximal end 28 and distal end 30 such that, when the implant insert 26 is releasably secured in the first lumen 24A, the distal end 30 is proximal to the distal end 18 of the guide body 12. According to other aspects (not shown), the elongate length of the implant insert 26 may be configured such that, when the implant insert 26 is releasably secured in the first lumen 24A, the distal end 30 is flush with the distal end 18 of the guide body 12.

In some implementations, the compression member 36 of the implant insert 26 may be configured to remain compressed when the pin 49 is located at the closed end portion 62 of the transverse channel portion 58 so as to create additional friction to be overcome by the practitioner to move the coupler 38 back to the first position. Additionally or alternatively, the closed end portion 62 of the transverse channel portion 58 may be positioned closer to the proximal end 28 of the implant insert 26 than a part of the transverse channel portion 58 immediately adjacent the closed end portion 62, creating a further element of resistance to rotation of the coupler 38 to the first position.

Referring again to FIG. 2, the glenoid drill guide 10 may additionally or alternatively include one or more bone block engagement inserts 70 each configured to be removably secured in a different one of the lumens 24 of the guide body 12. In the illustrated example, the glenoid drill guide 10 includes a first bone block engagement insert 70A configured to be releasably secured in the first lumen 24A, or more particularly in the lumen 32 of the implant insert 26 when the implant insert 26 is releasably secured in the first lumen 24A, and a second bone block engagement insert 70B configured to be releasably secured in the second lumen 24B. Each bone block engagement insert 70 may generally be configured to accommodate and guide a K-wire into a coupled bone bock and glenoid bone, such as to temporarily pin the bone block in place against the glenoid bone in preparation of further steps of the procedure. The first bone block engagement insert 70A may thus be configured to adapt the lumen 32 of the implant insert 26 to accommodate and guide a K-wire into a coupled bone block and glenoid bone when the implant insert 26 is releasably secured in the first lumen 24A, and the second bone block engagement insert 70B may be configured to adapt the second lumen 24B of the guide body 12 to accommodate and guide a K-wire into a coupled bone block and glenoid bone.

FIG. 7 illustrates an exemplary configuration of the first bone block engagement insert 70A. As shown in the illustrated example, the first bone block engagement insert 70A may have a proximal end 72 and a distal end 74, and may define a lumen 76 extending between the proximal end 72 and distal end 74 of the first bone block engagement insert 70A. The lumen 76 may define a longitudinal axis extending between the proximal end 72 and distal end 74, and may be open at the proximal end 72 and distal end 74 for passing a K-wire through the lumen 76 and into a coupled bone block and glenoid bone when the implant insert 26 is releasably secured in the first lumen 24A and the first bone block engagement insert 70A is releasably secured in the lumen 32 of the implant insert 26. The lumen 76 of the first bone block engagement insert 70A may have a transverse cross-section that is perpendicular to and generally uniform along the longitudinal axis of the lumen 76. The transverse cross-section of the lumen 76 may be sized and/or shaped for enabling longitudinal movement of a K-wire through the lumen 76 while limiting transverse movement of the K-wire within the lumen 76.

The first bone block engagement insert 70A may include a handle 78, a coupler 80, and a stem 82. The handle 78 may be disposed at the proximal end 72 of the first bone block engagement insert 70A, and may define an interface to be gripped by a practitioner to move the first bone block engagement insert 70A relative to the guide body 12, as described in more detail below. In the illustrated example, the handle 78 is realized as an enlarged head with a knurled surface.

The coupler 80 of the first bone block engagement insert 70A may be positioned distal to the handle 78, and may be configured to releasably secure the first bone block engagement insert 70A in the lumen 32 of the implant insert 26 when the implant insert 26 is releasably secured in the first lumen 24A. The stem 82 of the first bone block engagement insert 70A may extend distally from the coupler 80 and terminate at the distal end 74 of the first bone block engagement insert 70A.

Referring now to FIGS. 6 and 7, in some implementations, the coupler 80 of the first bone block engagement insert 70A may include a compression member 84, which may be realized an O-ring disposed in a groove 86 formed on an outer facing surface 88 of the first bone block engagement insert 70A that is stepped radially outward from the stem 82. The lumen 32 of the implant insert 26 may have an enlarged proximal portion 90 for receiving the coupler 80 of the first bone block engagement insert 70A when fully seated in the lumen 32 of the implant insert 26, and may include a distal portion 91 for receiving the stem 82 of the first bone block engagement insert 70A when fully seated in the lumen 32 of the implant insert 26. Surfaces of the implant insert 26 defining the enlarged proximal portion 90 of the lumen 32 may be sized to compress the compression member 84 of the coupler 80 when the coupler 80 is received in the enlarged proximal portion 90, thereby creating a friction that resists removal of the first bone block engagement insert 70A from the lumen 32. The first bone block engagement insert 70A may thus be removed from the lumen 32 by pulling on the handle 78 with sufficient force to overcome such friction.

It will be appreciated that each bone block engagement insert 70 of the glenoid drill guide 10, such as the second bone block engagement insert 70B, may have a similar configuration as that of the first bone block engagement insert 70A illustrated in FIGS. 6 and 7 and discussed above. Correspondingly, the second lumen 24B of the guide body 12 may have a similar configuration as that of the lumen 32 of the implant insert 26 and discussed above. In the foregoing description, components of each bone block engagement inserts 70 that correspond to those of the first bone block engagement insert 70A discussed above may thus be referenced using the same references numerals used above in connection with the first bone block engagement insert 70A.

Referring again to FIGS. 1 and 2, in some implementations, each of the bone block engagement inserts 70 of the glenoid drill guide 10 may include a distal portion 92 defining the distal end 74 of the bone block engagement insert 70 and configured to form at least a portion of the bone block engagement portion 20 disposed at the distal end 18 of the guide body 12 when the bone block engagement insert 70 is releasably secured relative to the guide body 12. For instance, the first bone block engagement insert 70A may be configured to be releasably secured in the lumen 32 of the implant insert 26 when the implant insert 26 is releasably secured in the first lumen 24A such that the distal portion 92 and the distal end 74 of the first bone block engagement insert 70A is positioned distal to the distal end 18 of the guide body 12, and the second bone block engagement insert 70B may be configured to be releasably secured in the second lumen 32B of the guide body 12 such that the distal portion 92 and distal end 74 of the second bone block engagement insert 70B is also positioned distal to the distal end 18 of the guide body 12.

The distal portion 92 of each releasably secured bone block engagement insert 70 that extends from the distal end 18 of the guide body 12 may be configured to engage a bone block to be secured to a glenoid bone, as described in more detail below. In some implementations, the bone block engagement insert 70 may be configured to be releasably secured relative to the guide body 12 such that the distal ends 74 of the bone block engagement inserts 70 extend distally from the distal end 18 of the bone block engagement insert 70 by a same distance. For instance, the distal ends 74 of the releasably secured bone block engagement inserts 70 may be configured to be located in a same plane distal to the distal end 18 of the guide body 12 and normal to the longitudinal axes of the lumens 24 of the guide body 12.

As previously described, in some examples, the first lumen 24A and not the second lumen 24B of the guide body 12 may be configured to receive an implant insert 26, which in turn may define a lumen 32 for receiving the first bone block engagement insert 70A. So as to enable the first and second bone block engagement inserts 70A, 70B to have a same elongated length and also extend a same distance from the distal end 18 of the guide body 12 when secured relative to the guide body 12 as described above, the length of the lumen 32 of the implant insert 26 extending between the proximal end 28 and the distal end 30 of the implant insert 26 may be equivalent to the length of the second lumen 24B of the guide body 12 extending between the proximal end 16 and distal end 18 of the guide body 12. Conversely, the length of the first lumen 24A of the guide body 12 extending between the proximal end 16 and the distal end 18 of the guide body 12 may be shorter than the length of the second lumen 24B. More specifically, the proximal end 16 of the guide body 12 may be configured such that the opening to the first lumen 24A at the proximal end 16 of the guide body 12 is distally offset from the opening to the second lumen 24B at the proximal end 16 of the guide body 12. The openings of the first and second lumens 24A, 24B at the distal end 18 of the guide body 12 may be longitudinally aligned, such that the openings lie in a same plane normal to the longitudinal axes of the first and second lumens 24A, 24B.

Referring again to FIG. 5C, the implant insert 26 may be configured such that when the implant insert 26 is releasably secured in the first lumen 24A of the guide body 12, the handle 34 of the implant insert 26 may extend proximally from the opening to the first lumen 24A in the proximal end 16 of the guide body 12 such that the opening to the lumen 32 in the proximal end 28 of the implant insert 26 is longitudinally aligned with the opening to the second lumen 24B in the proximal end 16 of the guide body 12. In other words, the opening to the lumen 32 in the proximal end 28 of the implant insert 26 and the opening to the second lumen 24B in the proximal end 16 of the guide body 12 may lie in a same plane normal to the longitudinal axes of the first and second lumens 24A, 24B. Consequently, when the first bone block engagement insert 70A is releasably secured in the lumen 32 of the implant insert 26 and the second bone block engagement insert 70B is releasably secured in the second lumen 24B, the distal ends 74 of the first and second bone block engagement inserts 70A, 70B may extend a same distance from the distal end 18 of the guide body 12.

Referring again to FIG. 7, in some implementations, the stem 82 of each bone block engagement insert 70 may be formed by an outer tube 96 coupled to and extending distally from the coupler 80, and an inner tube 98 partially fixed within the outer tube 96. The inner tube 98, which in some implementations may also be coupled to the coupler 80, may extend through a distal end 100 of the outer tube 96, with the portion of the inner tube 98 extending distally of the outer tube 96 forming the distal portion 92 of the bone block engagement insert 70. In some implementations, the outer tube 96 and inner tube 98 of each bone block engagement insert 70 may be configured so that, when the bone block engagement insert 70 is releasably secured relative to the guide body 12, the distal end 100 of the outer tube 96 is flush with or proximal to the distal end 18 of the guide body 12, and the distal portion 92 of the bone block engagement insert 70 formed by the inner tube 98 is distal to the distal end 18 of the guide body 12.

As described above, the first lumen 24A of the guide body 12 may be configured to accommodate passage of and guide an implant into a coupled bone guide and glenoid bone, and may be configured to releasably secure the implant insert 26 relative to the guide body 12. To this end, the first lumen 24A may have a transverse cross-section that is sized and/or shaped to accommodate receiving an implant and the implant insert 26 while also limiting transverse movement of the implant and implant insert 26 within the first lumen 24A. The second lumen 24B and the lumen 32 of the implant insert 26 may each be configured to accommodate and guide passage of a drill bit into a coupled bone guide and glenoid bone, and may be configured to releasably secure a bone block engagement insert 70 relative to the guide body 12. To this end, the second lumen 24B and lumen 32 of the implant insert 26 may each have a transverse cross-section that is sized and/or shaped to accommodate receiving a drill bit and bone block engagement insert 70 while also limiting transverse movement of the drill bit and bone block engagement insert 70 within the lumen 24B, 32. Further, the lumen 76 of each bone block engagement insert 70 may be configured to accommodate passage of a K-wire into a coupled bone block and glenoid bone. To this end, the lumen 76 of each bone block engagement insert 70 have a transverse cross-section that is sized and/or shaped to accommodate receiving a K-wire while also limiting transverse movement of the K-wire within the lumen 76.

As a corollary to the above configuration, the transverse cross-section of the first lumen 24A of the guide body 12 may be sized to encompass the transverse cross-section of the implant insert 26, the transverse cross-section of the lumen 32 of the implant insert 26 may be equivalent to the transverse cross-section of the second lumen 24B of the guide body 12; and the transverse cross-sections of the lumens 24B, 32 may each be sized to encompass the transverse cross-section of each bone block engagement insert 70.

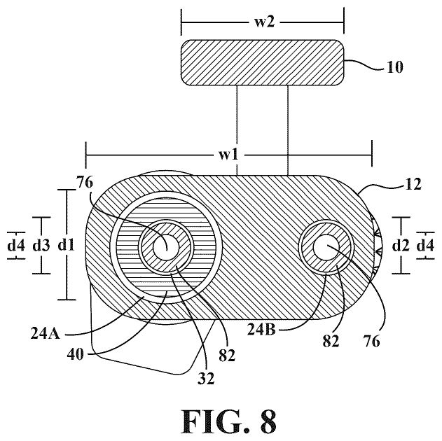

FIG. 8 illustrates an exemplary cross-section of the glenoid drill guide 10 taken along a lateral axis of the glenoid drill guide 10, which may be perpendicular to the longitudinal axes of the lumens 24 of the guide body 12. As shown in the illustrated example, the lumens 24, 32, and 76 and stems 40, 82 may each have a circular transverse cross-section. To facilitate the configuration of the glenoid drill guide 10 discussed above, the transverse cross-section of the first lumen 24A may have diameter d1 that is sufficient to receive the stem 40 of the implant insert 26 and an implant as described above, and that is greater than a diameter d2 of a transverse cross-section of the second lumen 24B of the guide body 12. The diameter d2 may be equal to a diameter d3 of a transverse cross-section of the lumen 32 of the implant insert 26. The diameter d2 and diameter d3 may each be sufficient to receive either the stem 82 of a bone block engagement insert 70 or a drill bit at a given time, and may each be greater than a diameter d4 of the transverse cross-section of the lumen 76 of each bone block engagement insert 70. The diameter d4 may be sufficient to receive a K-wire through the lumen 76.

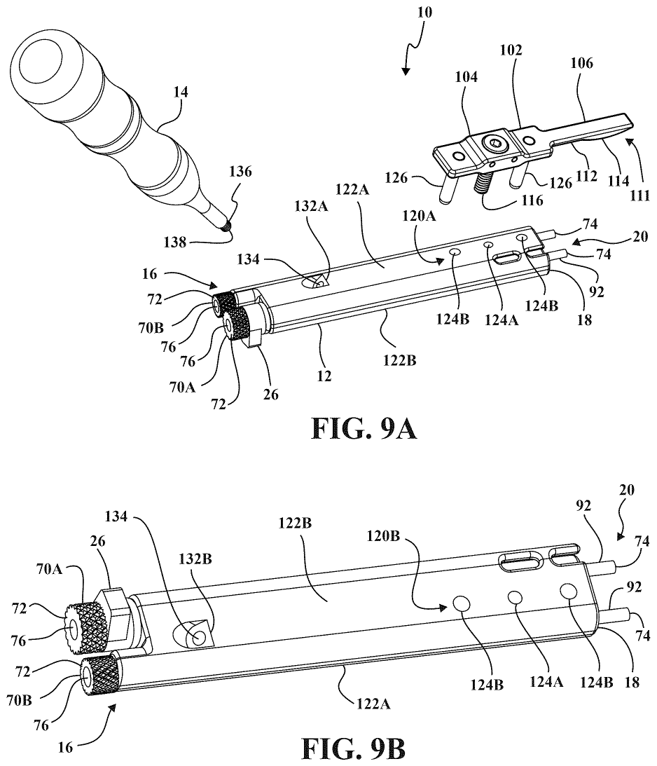

FIG. 9A illustrates the glenoid drill guide 10 with the handle 14 and an outrigger arm 102 that may be used with the glenoid drill guide 10 separated from the guide body 12. The outrigger arm 102 may be configured to help further stabilize a bone block coupled to the bone block engagement portion 20 of the glenoid drill guide 10, and/or to aid in alignment of the coupled bone block relative to the glenoid, or more particularly to the articular surface of the glenoid or to the surface of the glenoid bone underlying the articular surface, during a procedure. In some implementations, the outrigger arm 102 may be removeable so as to enable use of the glenoid drill guide 10 without the outrigger arm 102, such as may be preferrable by some practitioners.

The outrigger arm 102 may include a proximal portion 104 configured to be secured to the guide body 12 between the proximal end 16 and distal end 18 of the guide body 12, and a distal portion 106 configured to extend from the proximal portion 104 to beyond the distal end 18 of the guide body 12. The distal portion 106 may include a contact portion 111 facing inwards towards the bone block engagement portion 20 and configured to contact a surface of a bone block coupled to the bone block engagement portion 20 and/or a surface of the glenoid such as the glenoid articular surface formed by cartilage tissue residing on a bone of the glenoid. In some implementations, the contact portion 111 may include inward facing contact surfaces having varying profiles, such as to correspond to varying surfaces of the bone block and/or glenoid articular surface.

For instance, as shown in the illustrated example, the contact portion 111 may include a straight section 112 extending from the proximal portion 104 and a curved section 114 disposed distal to the straight section 112. In some implementations, the straight section 112 may be configured to contact a surface of a bone block when coupled to the bone block engagement portion 20 of the glenoid drill guide 10. In other implementations, the straight section 112 may not be configured to contact a coupled bone block, and rather primarily function to distally extend the curved section 114 from the bone block engagement portion 20. The curved section 114 may be configured to match the nominal curve of the glenoid articular surface or glenoid bone surface underlying the glenoid articular surface. Alternatively, according to other aspects, the curved section 114 may be configured with a radius of curvature that is less than that of the glenoid articular surface or glenoid bone surface underlying the glenoid articular surface. In either case, the curved section 114 may be configured to sit on the glenoid articular surface when a coupled bone block is properly aligned with the glenoid. In other examples, the contact portion 111 may omit the curved section 114, and/or may include contact surfaces with profiles of other shapes, such as a concave profile, a triangular profile, a sawtooth profile, or an L-shaped profile as some non-limiting examples.

The outrigger arm 102 may further include an adjustment mechanism 116 for adjusting the distance between the outrigger arm 102, or more particularly the contact portion 111 of the outrigger arm 102, and the bone block engagement portion 20 and/or lumens 24 defined by the guide body 12. In the illustrated example, the adjustment mechanism 116 is realized as a screw extending through the proximal portion 104 of the outrigger arm 102 and into the guide body 12. After a bone block is coupled to the bone block engagement portion 20 of the glenoid drill guide 10, the screw may be rotated to decrease the distance between the outrigger arm 102 and the bone block engagement portion 20 so that the contact portion 111 of the distal portion 106 of the outrigger arm 102 engages and applies a force on the bone block coupled to the bone block engagement portion 20. The contact portion 111 of the distal portion 106 of the outrigger arm 102 may thereby be configured in combination with the bone block engagement portion 20 to hold the bone block at the distal end 18 of the guide body 12, helping to stabilize the bone block on the bone block engagement portion 20 and ensure that the bone block does not fall off the bone block engagement portion 20 during the procedure.

Additionally or alternatively, in some implementations, the distal portion 106 of the outrigger arm 102 may be configured to flex relative to the proximal portion 104. In this case, the outrigger arm 102 may be adjusted to a desired distance prior to coupling the bone block to the bone block engagement portion 20. The desired distance may correspond to a distance in which the contact portion 111 of the distal portion 106 of the outrigger arm 102 will apply inward pressure on the bone block when coupled to the bone block engagement portion 20. Thereafter, when the bone block is coupled to the bone block engagement portion 20 of the glenoid drill guide 10, the bone block may cause the distal portion 106 of the outrigger arm 102 to flex away from the bone block engagement portion 20. The distal portion 106, which may be configured with a bias towards alignment with the proximal portion 104 of the outrigger arm 102, may thereby a apply force to a surface of the coupled bone block, achieving a similar stabilization effect.

As previously mentioned, the outrigger arm 102 may be removeable from the guide body 12, such as may be the preference of certain practitioners. Additionally, in some implementations, the orientation of the outrigger arm 102 relative to the bone block engagement portion 20 may be selectively configurable to accommodate different approaches, different practitioner preferences, and/or different patient anatomies (e.g., left side glenoid vs right side glenoid). To this end, the glenoid drill guide 10 may include two or more arm mounting interfaces 120 integral with the guide body 12, each configured to attach the outrigger arm 102 to a different portion of the guide body 12 such that the distal portion 106 of the outrigger arm 102 extends beyond the distal end 18 of the guide body 12 in a different orientation relative to the bone block engagement portion 20.

For instance, FIG. 9B illustrates the guide body 12 in a different orientation than that shown in FIG. 9A. As shown in FIGS. 9A and 9B, the guide body 12 may include opposed first and second sides 122A, 122B each extending along the length of the guide body 12 between the proximal end 16 and distal end 18 of the guide body 12. The guide body 12 may include a first arm mounting interface 120A disposed on the first side 122A of the guide body 12, and may include a second arm mounting interface 120B disposed on the second side 122B of the guide body 12. The first arm mounting interface 120A may be configured to couple the proximal portion 104 of the outrigger arm 102 to the first side 122A of the guide body 12 such that the distal portion 106 of the outrigger arm 102 extends beyond the distal end 18 of the guide body 12 from the first side 122A, and the second arm mounting interface 120B may be configured to couple the proximal portion 104 of the outrigger arm 102 to the second side 122B of the guide body 12 such that the distal portion 106 of the outrigger arm 102 extends beyond the distal end 18 of the guide body 12 from the second side 122B.

In some implementations, the longitudinal axes of the first and second lumens 24A, 24B of the guide body 12 may define a plane, with the first side 122A of the guide body 12 being disposed on one side of the plane, and the second side 122B of the guide body 12 being disposed on the other side of the plane. In some examples, each of the first and second sides 122A, 122B may also be perpendicular to the plane. The first arm mounting interface 120A may be configured to secure the proximal portion 104 of the outrigger arm 102 to the guide body 12 such that the distal portion 106 of the outrigger arm 102 extends beyond the distal end 18 of the guide body 12 on one side of the plane, namely the side of the plane corresponding to the first side 122A of the guide body 12, and the second arm mounting interface 120B may be configured to secure the proximal portion 104 of the outrigger arm 102 to the guide body 12 such that the distal portion 106 of the outrigger arm 102 extends beyond the distal end 18 of the guide body 12 on the other side of the plane, namely the side of the plane corresponding to the second side 122B of the guide body 12.

In some implementations, each arm mounting interface 120 may include a plurality of apertures 124 disposed adjacent the distal end 18 of the guide body 12. More specifically, each arm mounting interface 120 may include an aperture 124A for receiving the adjustment mechanism 116 of the outrigger arm 102 when coupled to the guide body 12. In the examples in which the adjustment mechanism 116 is realized as a screw, the aperture 124A may be threaded for receiving the threaded portion of the screw. In some implementations, the outrigger arm 102 may also include one or more alignment posts 126 extending inward towards the guide body 12 from the proximal portion 104 of the outrigger arm 102. In this case, each arm mounting interface 120 may also include one or more apertures 124B for receiving the alignment post(s) 126 of the outrigger arm 102 to ensure proper alignment of the distal portion 106 of the outrigger arm 102 relative to the bone block engagement portion 20. In the illustrated example, the outrigger arm 102 includes two alignment posts 126 on each side of the adjustment mechanism 116 such that the alignment post(s) 126 and adjustment mechanism 116 are arranged in a line, with the apertures 124 of each arm mounting interface 120 having a corresponding arrangement. In other aspects, the alignment post(s) 126 may be omitted.

Although described above as including a rotatable screw, in other aspects, the adjustment mechanism 116 for adjusting the height of the outrigger arm 102 relative to the bone block engagement portion 20 and/or lumens 24 defined by the guide body 12 may be realized by alternative configurations. For instance, in other aspects, the adjustment mechanism 116 may be realized as a ratchet-type mechanism. More specifically, and as one non-limiting example, the outrigger arm 102 may define one or more lumens each extending therethrough and configured to receive a ratchet arm coupled to and extending outwardly from the guide body 12 in a direction in which the outrigger arm 102 is adjustable. Each ratchet arm may define a series of teeth along its side, and the outrigger arm 102 may further include a pawl extending transversely into each lumen and configured to selectively engage the teeth of a received retched arm so as to hold the outrigger arm 102 at a desired distance from the bone block engagement portion 20 and/or lumens 24 defined by the guide body 12. The teeth may be angled such that the outrigger arm 102 may be moved towards the bone block engagement portion 20 and/or lumens 24 defined by the guide body 12 via a corresponding force being applied to the outrigger arm 102 by a user, but not able to be moved in the opposite direction unless a release mechanism of the outrigger arm 102 is actuated that moves the pawls out of their respective lumens.

Referring again to FIG. 8, in some implementations, a width w1 of the guide body 12 defined along its lateral axis may be greater than a corresponding width w2 of the outrigger arm 102 defined along its lateral axis. In this way, the practitioner can visualize portions of the bone block and glenoid that extend laterally beyond the outrigger arm 102 when using the glenoid drill guide 10 so as to help confirm the positioning of the bone block relative to the glenoid.

As previously mentioned, in some implementations, the orientation of the handle 14 relative to the guide body 12 may also be selectively configurable to accommodate different approaches, different practitioner preferences, and/or different patient anatomies (e.g., left side glenoid vs right side glenoid). For instance, referring FIGS. 4 and 9, the guide body 12 may include two or more handle mounting interfaces 132 integral with the guide body 12, each configured to releasably secure the handle 14 to the guide body 12 such that the handle extends outwardly from the guide body in a different orientation relative to the bone block engagement portion 20 and lumens 24 of the guide body 12. For instance, similar to the arm mounting interfaces 120, the guide body 12 may include a first handle mounting interface 132A disposed on the first side 122A of the guide body 12, and may include a second handle mounting interface 132B disposed on the second side 122B of the guide body 12. The first handle mounting interface 132A may be configured to releasable secure the handle 14 to the guide body 12 such that the handle 14 extends outwardly from the guide body 12 from the first side 122A of the guide body 12 and/or on a side of the plane defined by the longitudinal axes of the first and second lumens 24A, 24B of the guide body 12 corresponding to the first side 122A. The second handle mounting interface 132B may similarly be configured to releasable secure the handle 14 to the guide body 12 such that the handle 14 extends outwardly from the guide body 12 from the second side 122B of the guide body 12 and/or on a side of the plane corresponding to the second side 122B.

The handle mounting interface(s) 132 may be configured to secure the handle 14 to the guide body 12 using different techniques according to various aspects. As shown in the illustrated example, in some aspects, each handle mounting interface 132 may include a recessed aperture 134 configured to engage a coupling feature 136 disposed at a distal end 138 of the handle 14. In some implementations, the recessed aperture 134 may be threaded to correspond to threads of the coupling feature 136. In alternative implementations, the recessed aperture 134 may be configured to secure the coupling feature 136 via a snap fit engagement. In at least some implementations, the recessed aperture 134 may be angled relative to the guide body 12 so that the handle 14 extends both outwardly from and towards the proximal end 16 of the guide body 12.

As previously described, in some implementations, the first lumen 24A and not the second lumen 24B of the guide body 12 may be configured to guide an implant into a coupled bone block and glenoid bone. In this case, whether the guide body 12 is configured to guide an implant into a superior or inferior portion of the glenoid bone may be based on the selected orientations of the handle 14 and/or outrigger arm 102 relative to the lumens 24 of the guide body 12. Specifically, when the handle 14 and/or outrigger arm 102 are attached to the guide body 12 on one of the first and second sides 122A, 122B as described above, the first lumen 24A may be configured to be disposed on the superior portion of the glenoid bone during a procedure, and when the handle 14 and/or outrigger arm 102 are attached to the guide body 12 on the other of the first and second sides 122A, 122B as described above, the first lumen 24A may configured to be disposed on the inferior portion of the glenoid bone during the procedure. The practitioner may thus select whether the glenoid drill guide 10 is configured to aid in securing an implant into the superior or inferior portion of the glenoid bone based on which of the first and second sides 122A, 122B on which the handle 14 and/or outrigger arm 102 are disposed.

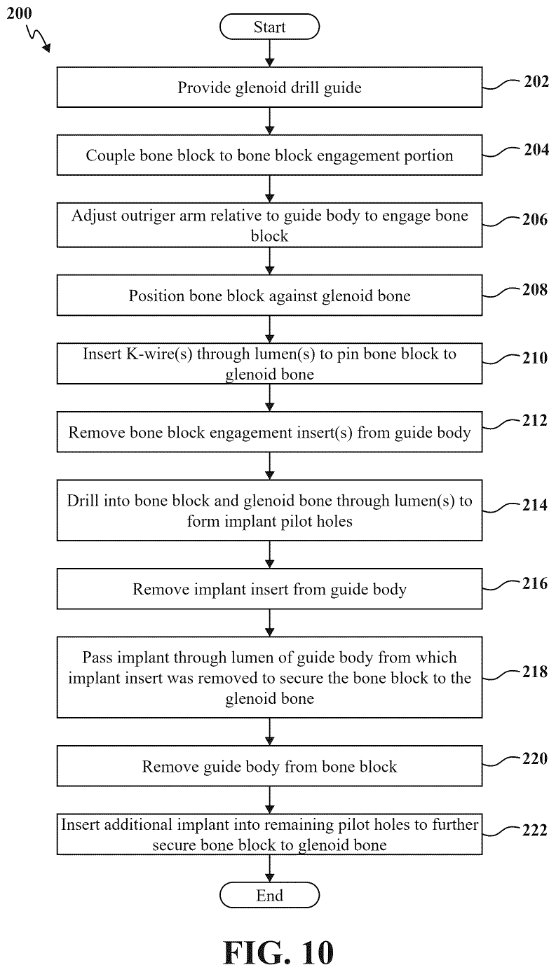

FIG. 10 illustrates a method 200 for repairing glenoid bone loss using the glenoid drill guide 10. As described herein, use of the glenoid drill guide 10 may assist the practitioner to achieve proper alignment of the bone block and implants. According to some aspects, the bone block may be aligned with respect to the glenoid articular surface. According to other aspects, the bone block may be aligned with respect to the surface of the glenoid bone underlying the articular surface. The bone block may be aligned with respect to the glenoid so the implants are positioned in desirable positions with respect to the articular surface and/or the glenoid bone surface underlying the articular surface.

In step 202, the glenoid drill guide 10 may be provided. Step 202 may include preparing the glenoid drill guide 10 as described above. For instance, the practitioner may releasably secure the implant insert 26 in the first lumen 24A of the guide body 12, releasably secure the first bone block engagement insert 70A in the lumen 32 of the secured implant insert 26, and releasably secure the second bone block engagement insert 70B in the second lumen 24B of the guide body 12.

Step 202 may also include securing the handle 14 to a handle mounting interface 132 integral with the guide body 12 and/or the outrigger arm 102 to an arm mounting interface 120 integral with the guide body 12, such as based on the patient anatomy being treated and/or the preferences of the practitioner. In some examples, the handle 14 and outrigger arm 102 may be secured to a same side 122 of the guide body 12, such as the first side 122A or the second side 122B, such that the handle 14 extends outwardly from such side 122 and towards the proximal end 16 of the guide body 12, and such that the distal portion 106 of the outrigger arm 102 extends beyond the distal end 18 of the guide body 12 from such side 122. Alternatively, one or more of the above preparation steps may occur elsewhere such as at the factory and/or prior to the practitioner receiving the glenoid drill guide 10.

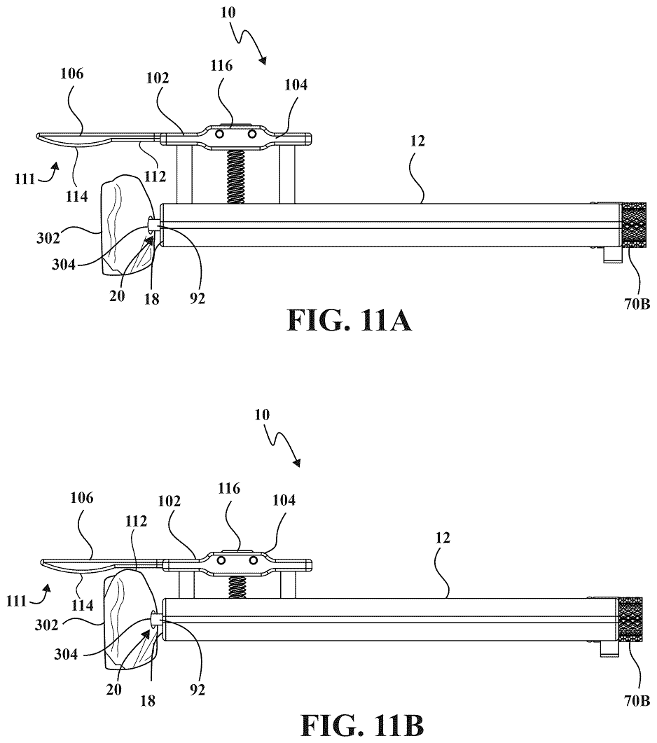

In step 204, a bone block may be coupled to the bone block engagement portion 20 of the guide body 12. FIG. 11A illustrates a bone block 302 that has been coupled to the bone block engagement portion 20 of the guide body 12 in accordance with step 204. The bone block 302 may be pre-drilled with a distinct hole 304 for the distal portion 92 of each of the bone block engagement inserts 70A, 70B that extends distally from the distal end 18 of the guide body 12. The bone block 302 may then be coupled to the bone block engagement portion 20 by maneuvering the glenoid drill guide 10 and the bone block 302 towards each other such that the distal portion 92 of each of the bone block engagement inserts 70A, 70B is received in a corresponding pre-drilled hole 304 of the bone block 302. The distal portion 92 of each of the bone block engagement inserts 70A, 70B may be pushed into its corresponding pre-drilled hole 304 until the bone block 302 contacts the distal end 18 of the guide body 12 or until the distal end 74 of at least one of the bone block engagement inserts 70A, 70B reaches a distal end of its corresponding pre-drilled hole 304. In some implementations, the distal portions 92 of the bone block engagement inserts 70A, 70B may form a friction fit with the pre-drilled holes 304 that function to retain the bone block 302 at the distal end 18 of the guide body 12 unless a sufficient force is applied to the bone block 302 or the glenoid drill guide 10 to overcome the friction fit.

In step 206, the outrigger arm 102 may be adjusted relative to the guide body 12 and the bone block engagement portion 20 so as to engage the coupled bone block 302. FIG. 11B illustrates the outrigger arm 102 having been adjusted in accordance with step 206 according to one aspect. The practitioner may operate the adjustment mechanism 116 to move the distal portion 106 of the outrigger arm 102 towards the lumens 24 of the guide body 12 and bone block engagement portion 20 so that the contact portion 111 of the outrigger arm 102, or more particularly the straight section 112 of the contact portion 111, contacts and applies a force on the bone block 302 in the direction of the bone block engagement portion 20. Such contact may be configured to further stabilize the bone block 302 on the bone block engagement portion 20.

Alternatively, in other implementations, the outrigger arm 102 may first be adjusted to a desired distance from the bone block engagement portion 20 of the glenoid drill guide 10, and then the bone block 302 may be coupled to the bone block engagement portion 20 as described above. During such coupling, the bone block 302 may cause the distal portion 106 to flex relative to the proximal portion 104 of the outrigger arm 102 and away from the bone block engagement portion 20 and lumens 24. The distal portion 106 may be biased towards alignment with the proximal portion 104, and may thus apply a force on the bone block 302 when in the flexed state that functions to stabilize the bone block 302 on the bone block engagement portion 20.

In yet further implementations, the bone block 302 may already be coupled to and/or the outrigger arm 102 adjusted when the glenoid drill guide 10 is provided in step 202. In further implementations, the outrigger arm 102 may be removed from the glenoid drill guide 10, such as according to the preferences of the practitioner, or may be positioned relative to the bone block engagement portion 20 and lumens 24 so as to maintain a desired offset between the outrigger arm 102 and coupled bone block 302, as described in more detail below.

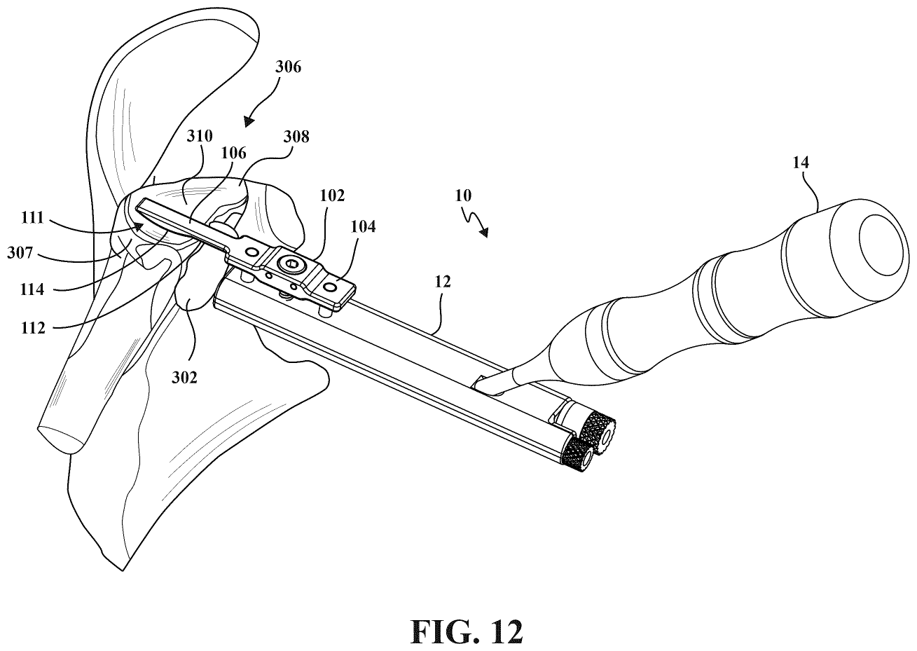

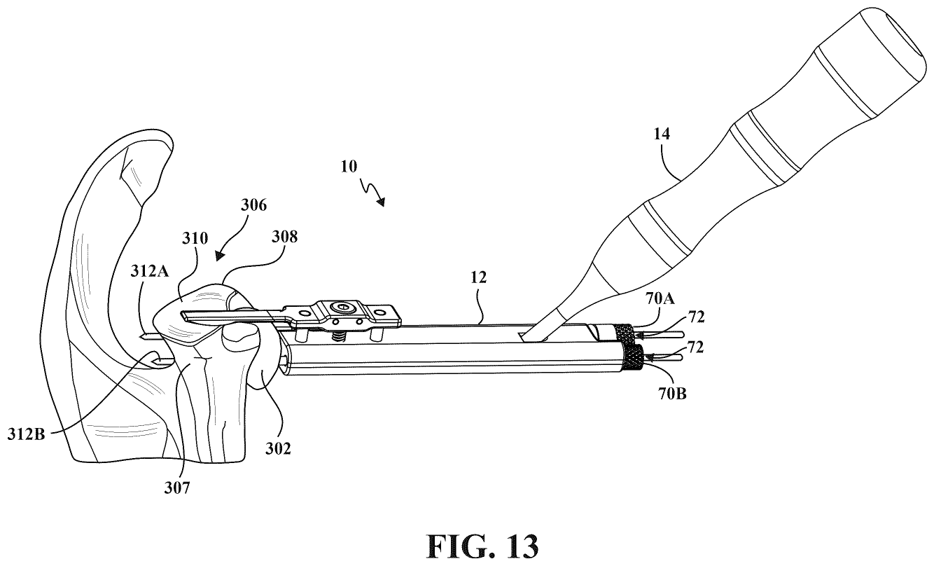

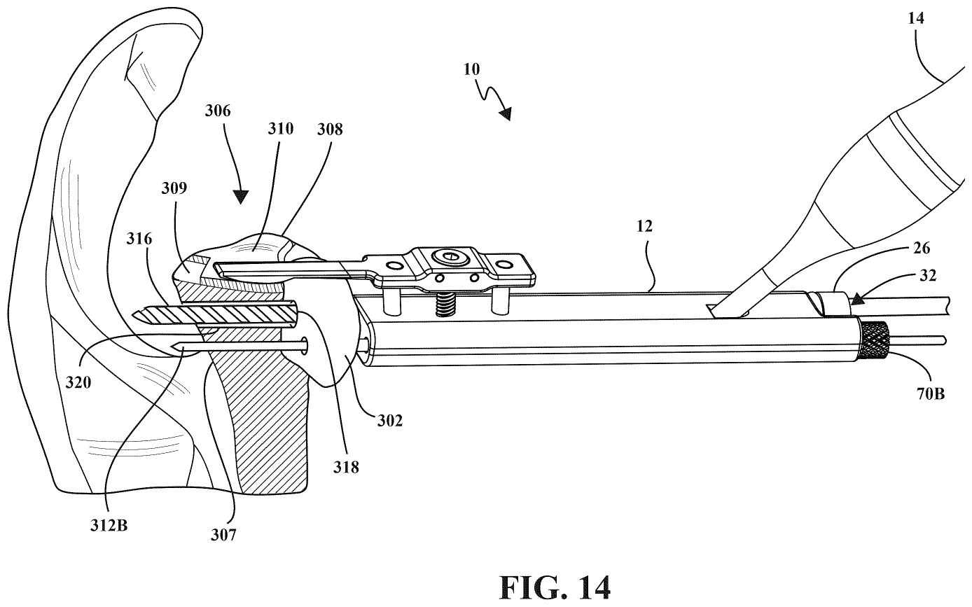

In step 208, the coupled and/or engaged bone block 302 may be positioned against a glenoid bone to be treated. FIG. 12 illustrates a glenoid 306 including a glenoid bone 307 to be treated for bone loss, with the coupled and engaged bone block 302 having been positioned against the glenoid bone 307 in accordance with step 208. The practitioner may grasp the handle 14 to maneuver the coupled and/or engaged bone block 302 into such position. FIG. 12 also illustrates cartilage tissue 308 of the glenoid 306. The cartilage tissue 308 may be disposed over an underlying surface 309 of the glenoid bone 307 (see, e.g., FIG. 14), and may form the articular surface 310 of the glenoid 306 for contacting the humeral head. When using the outrigger arm 102, the practitioner may position the bone block 302 by maneuvering the glenoid drill guide 10 such that the distal portion 106 of the outrigger arm 102, or more particularly the curved section 114 of the distal portion 106, sits on the articular surface 310 of the glenoid 306. This step may allow the practitioner to visualize and feel the two bony surfaces of the bone block 302 and glenoid bone 307, helping to ensure that they are flush before proceeding with drilling.

As described above, in some implementations, the outrigger arm 102 may be positioned such that the contact portion 111 of the outrigger arm 102, or more particularly the straight section 112 of the contact portion 111, contacts and applies a force on the bone block 302 in the direction of the bone block engagement portion 20. In some cases, however, it may be desirable to instead set the outrigger arm 102 to be separated from the coupled and/or engaged bone block 302 by an offset when positioning the bone block 302 against the glenoid bone 307 to be treated in step 208. In other words, the contact portion 111 of the outrigger arm 102, or more particularly the straight section 112 of the contact portion 111, may not touch the bone block 302. For instance, the offset may be selected such that when the contact portion 111 of the outrigger arm 102, or more particularly the curved section 114 of the contact portion 111, sits on the articular surface 310 of the glenoid 306, the bone block 302 may be disposed in a desired position relative to the underlying surface 309 of the glenoid bone 307 covered with the cartilage tissue 308 forming the articular surface 310, such as a position in which a surface of the bone block 302 facing the outrigger arm 102 is aligned with the underlying surface 309 of the glenoid bone 307. In other words, if the layer of cartilage tissue 308 forming the articular surface 310 is relatively thick, such offset may be selected so that when the contact portion 111, or more particularly the curved section 114 of the contact portion 111, sits on the articular surface 310, the bone block 302 may be aligned with the underlying surface 309 of glenoid bone 307 beneath the cartilage tissue 308 forming the articular surface 310, which may have a similar curvature to that of the articular surface 310.

When used, the outrigger arm 102 may also provide a visual aid that helps the practitioner to visualize the angle of the guide body 12 and the coupled and/or engaged bone block 302 relative to the glenoid 306, or more particularly the underlying surface 309 of the glenoid bone 307 and/or the glenoid articular surface 310, and correspondingly the pose in which instruments and/or implants may be guided through the bone block 302 and into the glenoid bone 307 by the glenoid drill guide 10. The outrigger arm 102 may also help ensure that the inserted implants are not too medial or lateral relative to the glenoid bone 307, and/or that the inserted implants are at a desired angle with respect to the articular surface 310 and/or the underlying surface 309 of the glenoid bone 307.

In step 210, one or more K-wires may be inserted through one or more of the lumens 76 of the bone block engagement inserts 70 and drilled into the bone block 302 and glenoid bone 307 to pin the bone block 302 in place against the glenoid bone 307. FIG. 13 illustrates the glenoid drill guide 10 with a first K-wire 312A having been inserted into the bone block 302 and glenoid bone 307 through the lumen 76 of the first bone block engagement insert 70A, and a second K-wire 312B having been inserted into the bone block 302 and glenoid bone 307 through the lumen 76 of the second bone block engagement insert 70B. In alternative examples, only a single K-wire 312 may be inserted into the bone block 302 and glenoid bone 307 via the glenoid drill guide 10. For instance, the first K-wire 312A may be inserted through the lumen 76 of the first bone block engagement insert 70A, or the second K-wire 312B may be inserted through the lumen 76 of the second bone block engagement insert 70B, but not both. In yet further examples, no K-wires may be used, in which case step 210 may be omitted from the method 200.

In step 212, at least one bone block engagement insert 70 may be removed from the guide body 12. For instance, the practitioner may remove the first bone block engagement insert 70A from the guide body 12 by pulling on the handle 78 of the first bone block engagement insert 70A to slide the first bone block engagement insert 70A out of the lumen 32 of the implant insert 26, and further over a proximal end of the first K-wire 312A if previously inserted through the lumen 76 of the first bone block engagement insert 70A as described above relative to step 210. Additionally or alternatively, the practitioner may remove the second bone block engagement insert 70B from the guide body 12 by pulling on the handle 78 of the second bone block engagement insert 70B to slide the second bone block engagement insert 70B out of the second lumen 24B of the guide body 12, and further over a proximal end of the second K-wire 312B if previously inserted through the lumen 76 of the second bone block engagement insert 70B as described above relative to step 210.

In step 214, a drill may be inserted through the guide body 12 to drill at least one pilot hole in the bone block 302 and the glenoid bone 307. For example, FIG. 14 illustrates a cannulated drill 316 having been inserted through the lumen 32 of the implant insert 26, from which the first bone block engagement insert 70A was previous removed, over the inserted first K-wire 312A, and into the bone block 302 and glenoid bone 307 to form an implant pilot hole 318 in the bone block 302 and an implant pilot hole 320 in the glenoid bone 307. The inserted first and second K-wires 312A, 312B may help keep the bone block 302 aligned with the glenoid bone 307 during drilling, preventing the bone block 302 from twisting.

As mentioned above relative to step 210, in some implementations, the step of inserting the first K-wire 312A through the lumen 76 of the first bone block engagement insert 70A may be omitted. In this case, step 214 may still include inserting the drill 316 through the lumen 32 of the implant insert 26 to form the implant pilot holes 318, 320, but optionally without the drill 316 being cannulated. If previously inserted in step 210, the second K-wire 312B, such as in combination with the outrigger arm 102 contacting the bone block 302 as described above, may continue to help keep the bone block 302 aligned with the glenoid bone 307 during drilling through the lumen 32 of the implant insert 26 without the first K-wire 312A being disposed therein.

During step 214, the practitioner may by design be able to perceive the angle of the guide body 12 relative to the glenoid articular surface 310 and/or the underlying surface 309 of the glenoid bone 307 via visualization of the outrigger arm 102 relative to the glenoid 306, or more particularly relative to the articular surface 310 and/or glenoid bone 307. Such perceived angle may help the practitioner to ensure the implant pilot holes 318, 320 are drilled at a generally parallel or other desired angle relative to the glenoid articular surface 310 and/or underlying surface 309.

FIG. 14 illustrates the drill 316 having been placed through the glenoid bone 307 bi-cortically to form the implant pilot holes 318, 320. In other implementations, the implant pilot holes 318, 320 may be formed by drilling through a single cortex of the glenoid bone 307.

In some cases, no further implant pilot holes may be desired. In alternative examples, the drill 316 may be drilled through the glenoid bone 307, either through a single cortex or bi-cortically, so as to form one or more additional implant pilot holes in each of the bone block 302 and glenoid bone 307. Specifically, in addition to being inserted through the lumen 32 of the implant insert 26 to form the implant pilot holes 318, 320, the drill 316 may also be inserted through the second lumen 24B of the guide body 12, from which the second bone block engagement insert 70B may have been previously removed in step 212, and through the bone block 302 and the glenoid bone 307 to form further implant pilot holes in the bone block 302 and the glenoid bone 307.

If the second K-wire 312B was previously inserted through the lumen 76 of the second bone block engagement insert 70B as described above relative to step 210, the drill 316 may be cannulated so as to also be slid over the inserted second K-wire 312B when forming the further implant pilot holes. Alternatively, the inserted second K-wire 312B may be removed from the bone block 302 and glenoid bone 307 through the second lumen 24B of the guide body 12 before drilling the further implant pilot holes in the bone block 302 and the glenoid bone 307 through the second lumen 24B of the guide body 12, such as after drilling the implant pilot holes 318, 320 through the lumen 32 of the implant insert 26 without the first K-wire 312A being disposed therein as described above. In this case, the drill 316 used to form the further implant pilot holes may not be cannulated.

In step 216, the implant insert 26 may be removed from the first lumen 24A of the guide body 12. For example, the practitioner may remove the implant insert 26 from the first lumen 24A by rotating the handle 34 of the implant insert 26 so as to transition the coupler 38 from the second position to the first position as described above, and thereafter pull on the handle 34 to remove the implant insert 26 out of the first lumen 24A. If the first K-wire 312A was previously inserted through the lumen 76 of the first bone block engagement insert 70A as described above relative to step 210, step 216 may also include pulling the implant insert 26 over a proximal end of the inserted first K-wire 312A.

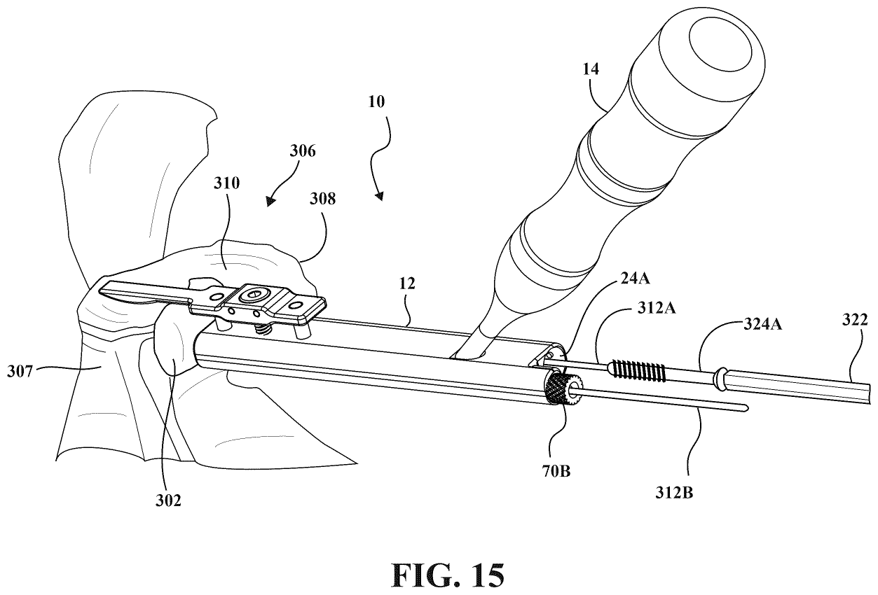

In step 218, an implant may be passed through the first lumen 24A from which the implant insert 26 was previously removed and into the implant pilot holes 318, 320 formed in the bone block 302 and glenoid bone 307 to secure the bone block 302 to the glenoid bone 307. FIG. 15 illustrates an example in which a driver 322 is being used to pass a first implant 324A through the first lumen 24A and into the implant pilot holes 318, 320. As shown in the illustrated example, the first implant 324A may be a cannulated screw that is inserted over the inserted first K-wire 312A and through the first lumen 24A for placement in the bone block 302 and the glenoid bone 307.

In alternative implementations, inserting the first K-wire 312A in step 210 may have been omitted, or the first K-wire 312A may be removed from the bone block 302, glenoid bone 307, and first lumen 24A prior to passing the first implant 324A through the first lumen 24A and into the bone block 302 and glenoid bone 307 in step 218. In either scenario, the first implant 324A may be realized as a solid screw. Assuming a second K-wire 312B was previously inserted into the bone block 302 and glenoid bone 307 in step 210 and has not yet been removed, the second K-wire 312B may remain inserted in the bone block 302 and glenoid bone 307 during step 218 to provide stability during insertion of the first implant 324A. In yet further examples, rather than a screw, the first implant 324A may be realized as an all-suture anchor such as Applicant's ICONIX anchor, or another type of implant, such as Applicant's KNOTILUS anchor that does not have screw threads.

In step 220, the guide body 12, including the second bone block engagement insert 70B if not previously removed, may be removed from the bone block 302 and glenoid bone 307. If any previously inserted K-wires 312 remain inserted in the bone block 302 and glenoid bone 307, the guide body 12 may also be removed over such K-wires 312. Alternatively, any previously inserted and remaining K-wires 312 may be removed prior to removal of the guide body 12.

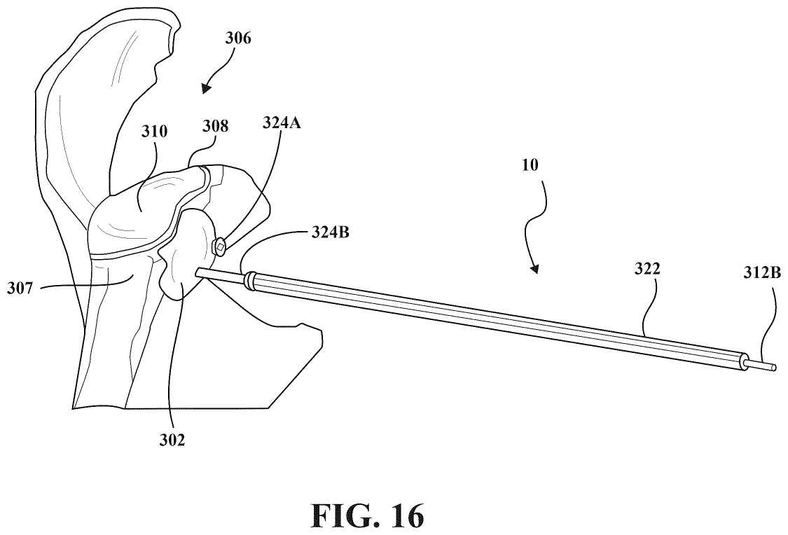

In step 222, a second implant 324B may be inserted in the bone block 302 and glenoid bone 307 to further secure the bone block 302 to the glenoid bone 307. As discussed above, in some implementations, the second K-wire 312B may remain inserted in the bone block 302 and glenoid bone 307 after removal of the guide body 12 in step 220. Assuming implant pilot holes were previously formed over the second K-wire 312B in step 214, the second implant 324B may be cannulated so as to be inserted over the second K-wire 312B and into the implant pilot holes. FIG. 16 illustrates the second implant 324B being inserted into the bone block 302 and the glenoid bone 307 by the driver 322 after removal of the guide body 12 and over the inserted second K-wire 312B.

Alternatively, if implant pilot holes were not previously formed in the bone block 302 and glenoid bone 307 over the inserted second K-wire 312B in step 214, such implant pilot holes may be formed after the guide body 12 has been removed by placing the cannulated drill 316 over the second K-wire 312B. This technique may allow the surgeon to make minor adjustments by hand to the position of the bone block 302 prior to forming the implant pilot holes for the second implant 324B and/or after insertion of the first implant 324A. As a result, the surgeon may also make minor adjustments to the angle of the implant pilot holes for the second implant 324B, and correspondingly to the angle of the second implant 324B, relative to the articular surface 310 as compared to forming such implant pilot holes over the second K-wire 312B through the guide body 12. Following forming the implant pilot holes in the bone block 302 and glenoid bone 307 over the second K-wire 312B in this way, the second implant 324B may be driven into the implant pilot holes using the driver 322 over the second K-wire 312B, in which case the second implant 324B may be cannulated. Alternatively, the second K-wire 312B may be removed following the forming of the implant pilot holes over the second K-wire 312B after the guide body 12 has been removed, in which case the second implant 324B may be solid.

As yet a further example, the guide body 12 may have been removed from the bone block 302 and glenoid bone 307 without any implant pilot holes for the second implant 324B having been formed in the bone block 302 and glenoid bone 307, with the second K-wire 312B also having been previously removed from the bone block 302 and glenoid bone 307 as described above, or having been omitted from the method 200 entirely. In this case, following removal of the guide body 12 from the bone block 302 and glenoid bone 307, the surgeon may proceed to form implant pilot holes in the bone block 302 and glenoid bone 307 for the second implant 324B “free-hand” using the drill 316, and then proceed to step 222 to insert the second implant 324B into the implant pilot holes. This technique may allow the surgeon even further latitude to adjust the bone block 302 by hand before forming the implant pilot holes for the second implant 324B, such as by rotating the bone block 302 relative to a longitudinal axis of the previously inserted first implant 324A. Further according to this example, it will be appreciated that the drill 316 and/or the second implant 324B may not be cannulated to effect this step of the procedure.

In another example, step 222 may include inserting the second implant 324B into the bone block 302 and glenoid bone 307 without implant pilot holes having been previously formed in the bone block 302 and glenoid bone 307 for the second implant 324B using the drill 316. For instance, following removal of the guide body 12 from the bone block 302 and glenoid bone 307, and optionally adjusting the bone block 302 by hand as described above, the surgeon may place the distal end of the second implant 324B into the pre-drilled hole 304 formed in the bone block 302 for receiving the distal portion 92 of the of the second bone block engagement insert 70B as described above, and proceed to drive the second implant 324B into the bone block 302 and glenoid bone 307 through the pre-drilled hole 304 using the driver 322. In this case, assuming the second K-wire 312B is still inserted in the bone block 302 and glenoid bone 307, the second implant 324B may be cannulated to as to be slide over the second K-wire 312B. Alternatively, if the second K-wire 312B was already removed or omitted form the method 200 as described above, the second implant 324B may be solid.

In the above-described examples, the first lumen 24A of the guide body 12 may be utilized to help guide insertion of a first implant 324A into the bone block 302 and glenoid bone 307 via implant pilot holes 318, 320 formed in the bone block 302 and glenoid bone 307 using the glenoid drill guide 10. In other implementations, after drilling the implant pilot holes 318, 320 in the bone block 302 and glenoid bone 307, the practitioner may remove the glenoid drill guide 10 from the bone block 302, and then proceed to secure the first implant 324A into the implant pilot holes 318, 320 without using the glenoid drill guide 10. If the second implant 324B is to be used, the practitioner may also proceed to insert the second implant 324B into the bone block 302 and glenoid bone 307 according to one of the implementations described above.

The above disclosure provides varying examples in which the first implant 324A may be canulated to accommodate insertion over the first K-wire 312A, and other examples in which the first implant 324A may be solid, in which case the first K-wire 312A may have been removed prior to insertion of the first implant 324A or insertion of the first K-wire 312A may have been omitted. The above disclosure also describes examples in which the second implant 324B may be cannulated or solid under similar conditions. In some implementations, the first implant 324A and the second implant 324B may both be cannulated, and thus may each be inserted into the bone block 302 and glenoid bone 307 according to one of the cannulated implementations described above. Alternatively, the first implant 324A and the second implant 324B may both be solid, and thus may each be inserted into the bone block 302 and glenoid bone 307 according to one of the non-cannulated implementations described above.

In other implementations, the first implant 324A may be one cannulated or solid, and the second implant 324B may be the other of cannulated or solid. In this way, the cannulated one of the first and second implants 324A, 324B may be inserted into the bone block 302 and glenoid bone 307 over one of the first and second K-wires 312A, 312B, such as according to one of the cannulated implementations described above, and the solid one of the first and second implants 324A, 324B may be inserted into the bone block 302 and glenoid bone 307 without being passed over the other of the first and second K-wires 312A, 312B such as according to one of the non-cannulated implementations described above.

The glenoid drill guides and methods described herein provide several advantages over the conventional techniques for repairing glenoid bone loss. First, a glenoid drill guide as disclosed herein may be configured to directly engage and retain the bone block at the guide's distal end, which allows the surgeon to maneuver the bone block smoothly and effortlessly to the repair site for visualizing the bone block against the scapular neck. Even without utilization of an outrigger arm, the practitioner can visualize and feel the two bony surfaces to help ensure they are flush before drilling. Second, if desired, the customizable outrigger arm can be used to help align the bone block to the glenoid, such as by helping to ensure that the bone block is aligned with the glenoid articular surface and/or glenoid bone surface underlying the articular surface, and/or by helping to ensure that the bone block and/or implants are not too medial or lateral. Third, the practitioner can visualize the angle of the drill guide body relative to the glenoid articular surface to aid in drilling holes that are parallel to or at the surgeon's desired position with respect to the glenoid articular surface and/or underlying glenoid bone surface. For example and as described hereinabove, according to various aspects, the surgeon may use a glenoid drill guide as described herein to position and orient the bone block so it is aligned with the glenoid bone surface underlying the articular surface instead of the articular surface. After positioning the bone block against the glenoid bone in accordance with such alignment, the angle of the drill guide body relative to the glenoid articular surface and/or glenoid bone may serve as a visual aid to the practitioner of the angle of one or more holes drilled into to the glenoid bone using the glenoid drill guide relative to the underlying glenoid bone surface. Fourth, if desired, the customizable outrigger arm can also aid the surgeon in drilling holes generally parallel or at another desired angle relative to the glenoid articular surface since the arm may be touched off on the glenoid further posteriorly and further pronounces any angle mismatch between the glenoid articular surface and drill guide body. Fifth, the glenoid drill guide may be modular and equipped with removable inserts that keep the K-wires, drill bits and implants aligned and at the appropriate angle relative to the glenoid. By following the examples disclosed herein, practitioners can achieve accurate and reliable placement of bone blocks and implants, ensuring optimal repair of glenoid bone loss.

The glenoid drill guides described herein may be reusable, and may thus be formed of a material (e.g., stainless steel) that lends to reprocessing following a given procedure. Reprocessing of the glenoid drill guide 10 according to one exemplary aspect may include disassembling the glenoid drill guide 10 into its individual components (e.g., guide body 12, handle 14, implant insert 26, bone block engagement inserts 70A, 70B, outrigger arm 102, and compression members 36, 84), and then thoroughly cleaning and sterilizing each separate component. Cleaning may involve manual scrubbing or the use of ultrasonic cleaners to remove any debris or contaminants. Sterilization may be performed following cleaning, and may include placing the components in an autoclave, which may employ high-pressure steam to eliminate any remaining microorganisms on the components. The components of the glenoid drill guide 10 may then be reassembled as described herein in preparation for a next procedure. In some implementations, rather than being cleaned and/or sterilized, the removed compression members 36, 84 may be disposed of and replaced with new compression members 36, 84.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It will be understood that one or more steps within a method may be executed in a different order, may be executed serially or concurrently, and/or may include more or fewer steps than those illustrated and/or described above according to various aspects. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

Clauses for additional protection:
I. A glenoid drill guide comprising:
   a guide body having proximal and distal ends and defining first and second lumens each extending between the proximal and distal ends; first and second bone block engagement inserts each having proximal and distal ends, the first bone block engagement insert defining a first lumen extending between the proximal and distal ends of the first bone block engagement insert, the second bone block engagement insert defining a second lumen extending between the proximal and distal ends of the second bone block engagement insert, and the distal ends of the first and second bone block engagement inserts being configured to engage a bone block to be secured to a glenoid bone; and an implant insert having proximal and distal ends and defining a lumen extending between the proximal and distal ends of the implant insert,
   wherein the implant insert is configured to be removably secured in the first lumen such that the distal end of the implant insert is proximal to or flush with the distal end of the guide body, wherein the first bone block engagement insert is configured to be removably secured in the lumen of the implant insert when the implant insert is secured in the first lumen of the guide body such that the distal end of the first bone block engagement insert is distal to the distal end of the guide body, and wherein the second bone block engagement insert is configured to be removably secured in the second lumen of the guide body such that the distal end of the second bone block engagement insert is distal to the distal end of the guide body.
II. The glenoid drill guide of claim I, wherein a diameter of the first lumen of the guide body is greater than a diameter of the second lumen of the guide body.
III. The glenoid drill guide of claim I or II, wherein the first lumen and not the second lumen of the guide body is sized to accommodate passage of an implant for securing the bone block to the glenoid bone.
IV. The glenoid drill guide of any one of claims I-III, wherein a diameter of the lumen of the implant insert is equal to a diameter of the second lumen of the guide body.
V. The glenoid drill guide of any one of claims I-IV, further comprising an outrigger arm having a proximal portion and a distal portion, the proximal portion secured to the guide body between the proximal and distal ends of the guide body, and the distal portion extending from the proximal portion to beyond the distal end of the guide body, wherein the distal portion is configured in combination with the distal ends of the first and second bone block engagement inserts to retain the bone block at the distal end of the drill guide.
VI. The glenoid drill guide of claim V, further comprising an adjustment mechanism for adjusting a distance between the outrigger arm and the first and second lumens of the guide body.
VII. The glenoid drill guide of claim VI, wherein the adjustment mechanism comprises a screw extending through the proximal portion of the outrigger arm and into the guide body.
VIII. The glenoid drill guide of claim VI or VII, wherein the first lumen of the guide body defines a first longitudinal axis, the second lumen of the guide body defines a second longitudinal axis, the first and second longitudinal axes collectively define a plane, and the adjustment mechanism is configured to move the outrigger arm along an axis transverse to the plane.
IX. The glenoid drill guide of any one of claims V-VIII, wherein the distal portion of the outrigger arm comprises a straight section and a curved section, the curved section being located distal to the straight section.
X. The glenoid drill guide of any one of claims V-IX, wherein the first lumen of the guide body defines a first longitudinal axis, the second lumen of the guide body defines a second longitudinal axis, the first and second longitudinal axes collectively define a plane, and the distal portion of the outrigger arm extends beyond the distal end of the guide body on a first side of the plane, the glenoid drill guide further comprising a handle extending outwardly from the guide body on the first side of the plane.
XI. The glenoid drill guide of any one of claims V-X, wherein the guide body comprises first and second arm mounting interfaces, each of the first and second arm mounting interfaces being configured to releasably secure the proximal portion of the outrigger arm to a different portion of the guide body such that the distal portion of the outrigger arm extends beyond the distal end of the guide body in a different orientation relative to the first and second lumens of the guide body.
XII. The glenoid drill guide of claim XI, wherein the first lumen of the guide body defines a first longitudinal axis, the second lumen of the guide body defines a second longitudinal axis, the first and second longitudinal axes collectively define a plane, the first arm mounting interface is configured to releasably secure the proximal portion of the outrigger arm to the guide body such that the distal portion of the outrigger arm extends beyond the distal end of the guide body on a first side of the plane, and the second arm mounting interface is configured to releasably secure the proximal portion of the outrigger arm to the guide body such that the distal portion of the outrigger arm extends beyond the distal end of the guide body on a second side of the plane.
XIII. The glenoid drill guide of any one of claims V-XII, wherein a width of the guide body is greater than a width of the outrigger arm.
XIV. The glenoid drill guide of any one of claims I-XIII, wherein the implant insert comprises a coupler moveable between a first position relative to the guide body in which the implant insert is able to slide longitudinally within the first lumen of the guide body and a second position relative to the guide body in which the implant insert is prevented from sliding longitudinally within the first lumen of the guide body.
XV. The glenoid drill guide of any one of claims I-XIV, wherein the first bone block engagement insert comprises a first compression member for releasably securing the first bone block engagement insert in the lumen of the implant insert, and the second bone block engagement insert comprises a second compression member for releasably securing the second bone block engagement insert in the second lumen of the guide body.
XVI. The glenoid drill guide of any one of claims I-XV, further comprising:
   a handle; and
   first and second handle interfaces integral with the guide body, the first handle interface being configured to releasably secure the handle to the guide body such that the handle extends outwardly from the guide body in a first orientation relative to the first and second lumens of the guide body, and the second handle interface being configured to releasable secure the handle to the guide body such that the handle extends outwardly from the guide body in a second orientation relative to the first and second lumens of the guide body that differs from the first orientation.
XVII. The glenoid drill guide of claim XVI, wherein the first lumen of the guide body defines a first longitudinal axis, the second lumen of the guide body defines a second longitudinal axis, the first and second longitudinal axes collectively define a plane, the first handle interface is configured to releasably secure the handle to the guide body such that the handle extends outwardly from the guide body on a first side of the plane, and the second handle interface is configured to releasably secure the handle to the guide body such that the handle extends outwardly from the guide body on a second side of the plane.
XVIII. A glenoid drill guide comprising:
   a guide body having proximal and distal ends and defining a lumen extending between the proximal and distal ends;
   a bone block engagement portion disposed at the distal end of the guide body and configured to engage a bone block to be secured to a glenoid bone; and
   an outrigger arm including a proximal portion and a distal portion, the proximal portion being secured to the guide body, and the distal portion extending from the proximal portion to beyond the distal end of the guide body,
   wherein the distal portion of the outrigger arm is configured in combination with the bone block engagement portion to retain the bone block at the distal end of the guide body, and
   wherein the outrigger arm is adjustable between a first distance from the bone block engagement portion and a second distance from the bone block engagement portion, the first distance being greater than the second distance.
XIX. The glenoid drill guide of claim XVIII, further comprising:
   a bone block engagement insert having proximal and distal ends and defining a lumen extending between the proximal and distal ends of the bone block engagement insert, the distal end of the bone block engagement insert being configured to engage the bone block; and
   an implant insert having proximal and distal ends and defining a lumen extending between the proximal and distal ends of the implant insert,
   wherein the implant insert is configured to be removably secured in the lumen of the guide body such that the distal end of the implant insert is proximal to or flush with the distal end of the guide body, and
   wherein the bone block engagement insert is configured to be removably secured in the lumen of the implant insert when the implant insert is secured in the lumen of the guide body such that the distal end of the bone block engagement insert is distal to the distal end of the guide body and forms the bone block engagement portion disposed at the distal end of the guide body.
XX. The glenoid drill guide of claim XIX, wherein the lumen of the drill guide and not the lumen of the implant insert is sized to accommodate passage of an implant for securing the bone block to the glenoid bone.
XXI. The glenoid drill guide of claim XIX or XX, wherein the implant insert comprises a coupler moveable between a first position relative to the guide body in which the implant insert is able to slide longitudinally within the lumen of the guide body and a second position relative to the guide body in which the implant insert is prevented from sliding longitudinally within the lumen of the guide body.
XXII. The glenoid drill guide of any one of claims XIX-XXI, wherein the bone block engagement insert comprises a compression member for releasably securing the bone block engagement insert in the lumen of the implant insert.
XXIII. The glenoid drill guide of any one of claims XVIII-XXII, further comprising a screw extending through the proximal portion of the outrigger arm and into the guide body, the screw configured to move the outrigger arm towards the bone block engagement portion when the screw is turned in a first direction and to move the outrigger arm away from the bone block engagement portion when the screw is turned in a second direction opposite the first direction.
XXIV. The glenoid drill guide of any one of claims XVIII-XXIII, wherein the distal portion of the outrigger arm comprises a straight section and a curved section, the curved section being located distal to the straight section.
XXV. The glenoid drill guide of any one of claims XVIII-XXIV, wherein the guide body comprises opposed first and second sides each extending between the proximal and distal ends of the guide body, and wherein the proximal portion of the outrigger arm is secured to the first side of the guide body, the glenoid drill guide further comprising a handle extending outwardly from the first side of the guide body.
XXVI. The glenoid drill guide of any one of claims XVIII-XXV, wherein the guide body comprises first and second arm mounting interfaces, each of the first and second arm mounting interfaces configured to releasably secure the proximal portion of the outrigger arm to a different portion of the guide body such that the distal portion of the outrigger arm extends beyond the distal end of the guide body in a different orientation relative to the bone block engagement portion.
XXVII. The glenoid drill guide of any one of claims XVIII-XXVI, further comprising:
   a handle; and
   first and second handle interfaces integral with the guide body, the first handle interface being configured to releasably secure the handle to the guide body such that the handle extends outwardly from the guide body in a first orientation relative to the bone block engagement portion, and the second handle interface being configured to releasably secure the handle to the guide body such that the handle extends outwardly from the guide body in a second orientation relative to the bone block engagement portion that differs from the first orientation.
XXVIII. The glenoid drill guide of claim XXVII, wherein the guide body comprises opposed first and second sides each extending between the proximal and distal ends of the guide body, the guide body further comprises first and second arm mounting interfaces each configured to releasably secure the proximal portion of the outrigger arm to the guide body such that the distal portion of the outrigger arm extends beyond the distal end of the guide body, the first handle interface and the first arm mounting interface are each disposed on the first side of the guide body, and the second handle interface and the second arm mounting interface are each disposed on the second side of the guide body.
XXIX. The glenoid drill guide of any one of claims XVIII-XXVIII, wherein a width of the guide body is greater than a width of the outrigger arm.
XXX. A method of repairing glenoid bone loss, the method comprising:
   providing a guide body having proximal and distal ends and defining first and second lumens each extending between the proximal and distal ends;
   securing an implant insert in the first lumen of the guide body such that a distal end of the implant insert is proximal to or flush with the distal end of the guide body, the implant insert defining a lumen extending between a proximal end and the distal end of the implant insert;
   securing a first bone block engagement insert in the lumen of the secured implant insert such that a distal end of the first bone block engagement insert is distal to the distal end of the guide body, wherein the first bone block engagement insert defines a first lumen extending between a proximal end and the distal end of the first bone block engagement insert;
   securing a second bone block engagement insert in the second lumen of the guide body such that a distal end of the second bone block engagement insert is distal to the distal end of the guide body, the second bone block engagement insert defining a second lumen extending between a proximal end and the distal end of the second bone block engagement insert;
   coupling a bone block to the distal ends of the secured first and second bone block engagement inserts;
   maneuvering the guide body with the coupled bone block to position the bone block against a glenoid bone;
   inserting a K-wire through the first lumen of the secured first bone block engagement insert or the second lumen of the secured second bone block engagement insert while the coupled bone block is positioned against the glenoid bone to pin the bone block to the glenoid bone;
   removing the first bone block engagement insert from the lumen of the secured implant insert after the K-wire is inserted;
   drilling into the bone block through the lumen of the secured implant insert after the first bone block engagement insert is removed;
   removing the implant insert from the first lumen of the guide body after the bone block is drilled; and
   passing an implant through the first lumen of the guide body to secure the bone block to the glenoid bone after the implant insert is removed.
XXXI. The method of claim XXX, wherein inserting the K-wire through the first lumen of the secured first bone block engagement insert or the second lumen of the secured second bone block engagement insert while the coupled bone block is positioned against the glenoid bone to pin the bone block to the glenoid bone comprises inserting the K-wire through the first lumen of the secured first bone block engagement insert.
XXXII. The method of claim XXXI, wherein the implant is cannulated, and wherein passing the implant through the first lumen of the guide body to secure the bone block to the glenoid bone after the implant insert is removed comprises passing the cannulated implant through the first lumen of the guide body over the inserted K-wire.
XXXIII. The method of claim XXXI or XXXII, wherein the K-wire is defined as a first K-wire, the method further comprising inserting a second K-wire through the second lumen of the secured second bone block engagement insert while the coupled bone block is positioned against the glenoid bone to pin the bone block to the glenoid bone.
XXXIV. The method of claim XXXI, wherein the K-wire is defined as a first K-wire, the method further comprising:
   inserting a second K-wire through the second lumen of the secured second bone block engagement insert while the coupled bone block is positioned against the glenoid bone to pin the bone block to the glenoid bone;
   removing the first K-wire from the first lumen of the secured first bone block engagement insert after the second K-wire is inserted; and
   passing the implant through the first lumen of the guide body to secure the bone block to the glenoid bone after the first K-wire and the implant insert are removed.
XXXV. The method of claim XXX, wherein inserting the K-wire through the first lumen of the secured first bone block engagement insert or the second lumen of the secured second bone block engagement insert while the coupled bone block is positioned against the glenoid bone to pin the bone block to the glenoid bone comprises inserting the K-wire through the second lumen of the secured second bone block engagement insert.
XXXVI. The method of any one of claims XXX-XXXV, further comprising:
   attaching a proximal portion of an outrigger arm to the guide body such that a distal portion of the outrigger arm extends beyond the distal end of the guide body; and
   adjusting a position the outrigger arm relative to the guide body to engage the coupled bone block.
XXXVII. The method of claim XXXVI, wherein the guide body includes opposed first and second sides each extending between the proximal and distal ends of the guide body, includes first and second arm mounting interfaces each configured to releasably secure the proximal portion of the outrigger arm to the guide body such that the distal portion of the outrigger arm extends beyond the distal end of the guide body, and includes first and second handle interfaces each configured to releasably secure a handle to the guide body such that the handle extends outwardly from the guide body, the first arm mounting interface and the first handle interface being disposed on the first side of the guide body, and the second arm mounting interface and the second handle interface being disposed on the second side of the guide body, the method further comprising:
   attaching the proximal portion of the outrigger arm to the first arm mounting interface such that the distal portion of the outrigger arm extends beyond the distal end of the guide body from the first side of the guide body; and
   attaching the handle to the first handle interface such that the handle extends outwardly from the guide body from the first side of the guide body.
XXXVIII. The method of any one of claims XXX-XXXVII, wherein securing the implant insert in the first lumen of the guide body comprises:
   inserting the implant insert in the first lumen of the guide body; and
   rotating the inserted implant insert relative to the guide body to engage a coupler of the implant insert that prevents longitudinal movement of the inserted implant insert relative to the first lumen of the guide body.
XXXIX. A method of repairing glenoid bone loss, the method comprising:
   providing a glenoid drill guide, the glenoid drill guide including a guide body, a bone block engagement portion disposed at a distal end of the guide body, and an outrigger arm, wherein the guide body defines a lumen extending between a proximal end and the distal end of the guide body, and wherein the outrigger arm includes a proximal portion secured to the guide body and a distal portion extending from the proximal portion to beyond the distal end of the guide body;
   coupling a bone block to the bone block engagement portion;
   adjusting a position the outrigger arm relative to the guide body such that the outrigger arm engages the coupled bone block;
   maneuvering the glenoid drill guide with the engaged bone block to position the bone block against a glenoid bone;
   inserting a K-wire through the lumen of the guide body while the engaged bone block is positioned against the glenoid bone to pin the bone block to the glenoid bone; and
   securing the bone block to the glenoid bone with an implant while the K-wire holds the bone block in place against the glenoid bone.
XL. The method of claim XXXIX, wherein the guide body includes opposed first and second sides each extending between the proximal and distal ends of the guide body, includes first and second arm mounting interfaces each configured to releasably secure the proximal portion of the outrigger arm to the guide body such that the distal portion of the outrigger arm extends beyond the distal end of the guide body, and includes first and second handle interfaces each configured to releasably secure a handle to the guide body such that the handle extends outwardly from the guide body, the first arm mounting interface and the first handle interface being disposed on the first side of the guide body, and the second arm mounting interface and the second handle interface being disposed on the second side of the guide body, the method further comprising:
   attaching the proximal portion of the outrigger arm to the first arm mounting interface such that the distal portion of the outrigger arm extends beyond the distal end of the guide body from the first side of the guide body; and
   attaching the handle to the second handle interface such that the handle extends outwardly from the guide body from the first side of the guide body.
XLI. A method of repairing glenoid bone loss, the method comprising:
   providing a guide body having proximal and distal ends and defining a lumen extending between the proximal and distal ends;
   securing a bone block engagement insert in the lumen of the guide body such that a distal end of the bone block engagement insert is distal to the distal end of the guide body, the bone block engagement insert defining a lumen extending between a proximal end and the distal end of the bone block engagement insert;
   coupling a bone block to the distal end of the secured bone block engagement insert;
   maneuvering the guide body with the coupled bone block to position the bone block against a glenoid bone;
   inserting a K-wire through the lumen of the secured bone block engagement insert while the coupled bone block is positioned against the glenoid bone to pin the bone block to the glenoid bone;
   removing the bone block engagement insert from the lumen of the guide body and over the inserted K-wire;
   drilling into the bone block through the lumen of the guide body and over the inserted K-wire after the bone block engagement insert is removed; and
   securing the bone block to the glenoid bone with an implant after the bone block is drilled.
XLII. The method of claim XLI, wherein the implant is cannulated, the method further comprising:
   securing an implant insert in the lumen of the guide body such that a distal end of the implant insert is proximal to or flush with the distal end of the guide body, the implant insert defining a lumen extending between a proximal end and the distal end of the implant insert, wherein securing the bone block engagement insert in the lumen of the guide body such that the distal end of the bone block engagement insert is distal to the distal end of the guide body comprises securing the bone block engagement insert in the lumen of the secured implant insert, wherein removing the bone block engagement insert from the lumen of the guide body over the inserted K-wire comprises removing the bone block engagement insert from the lumen of the implant insert, and wherein drilling into the bone block through the lumen of the guide body and over the inserted K-wire after the bone block engagement insert is removed comprises drilling into the bone block through the lumen of the implant insert and over the K-wire after the bone block engagement insert is removed;
   removing the implant insert from the lumen of the guide body and over the inserted K-wire after the bone block engagement insert is removed; and
   passing the cannulated implant through the lumen of the guide body and over the K-wire to secure the bone block in the glenoid bone after the implant insert is removed.

It will be further appreciated that the terms “include,” “includes,” and “including” have the same meaning as the terms “comprise,” “comprises,” and “comprising.” Moreover, it will be appreciated that terms such as “first,” “second,” “third,” and the like are used herein to differentiate certain structural features and components for the non-limiting, illustrative purposes of clarity and consistency. The terminology which has been used is intended to be in the nature of words of description rather than of limitation. Many modifications and variations are possible in light of the above teachings may be practiced otherwise than as specifically described.

What is claimed is:

1. A glenoid drill guide comprising:
a guide body having proximal and distal ends and defining first and second lumens each extending between the proximal and distal ends;
first and second bone block engagement inserts each having proximal and distal ends, the first bone block engagement insert defining a first lumen extending between the proximal and distal ends of the first bone block engagement insert, the second bone block engagement insert defining a second lumen extending between the proximal and distal ends of the second bone block engagement insert, and the distal ends of the first and second bone block engagement inserts being configured to engage a bone block to be secured to a glenoid bone; and
an implant insert having proximal and distal ends and defining a lumen extending between the proximal and distal ends of the implant insert,
wherein the implant insert is configured to be removably secured in the first lumen such that the distal end of the implant insert is proximal to or flush with the distal end of the guide body,
wherein the first bone block engagement insert is configured to be removably secured in the lumen of the implant insert when the implant insert is secured in the first lumen of the guide body such that the distal end of the first bone block engagement insert is distal to the distal end of the guide body, and
wherein the second bone block engagement insert is configured to be removably secured in the second lumen of the guide body such that the distal end of the second bone block engagement insert is distal to the distal end of the guide body.

2. The glenoid drill guide of claim 1, wherein a diameter of the first lumen of the guide body is greater than a diameter of the second lumen of the guide body.

3. The glenoid drill guide of claim 2, wherein the first lumen and not the second lumen of the guide body is sized to accommodate passage of an implant for securing the bone block to the glenoid bone.

4. The glenoid drill guide of claim 3, wherein a diameter of the lumen of the implant insert is equal to a diameter of the second lumen of the guide body.

5. The glenoid drill guide of claim 4, further comprising an outrigger arm having a proximal portion and a distal portion, the proximal portion secured to the guide body between the proximal and distal ends of the guide body, and the distal portion extending from the proximal portion to beyond the distal end of the guide body, wherein the distal portion is configured in combination with the distal ends of the first and second bone block engagement inserts to retain the bone block at the distal end of the drill guide.

6. The glenoid drill guide of claim 5, further comprising an adjustment mechanism for adjusting a distance between the outrigger arm and the first and second lumens of the guide body.

7. The glenoid drill guide of claim 6, wherein the adjustment mechanism comprises a screw extending through the proximal portion of the outrigger arm and into the guide body.

8. The glenoid drill guide of claim 7, wherein the first lumen of the guide body defines a first longitudinal axis, the second lumen of the guide body defines a second longitudinal axis, the first and second longitudinal axes collectively define a plane, and the adjustment mechanism is configured to move the outrigger arm along an axis transverse to the plane.

9. The glenoid drill guide of claim 8, wherein the distal portion of the outrigger arm comprises a straight section and a curved section, the curved section being located distal to the straight section.

10. The glenoid drill guide of claim 5, wherein the first lumen of the guide body defines a first longitudinal axis, the second lumen of the guide body defines a second longitudinal axis, the first and second longitudinal axes collectively define a plane, and the distal portion of the outrigger arm extends beyond the distal end of the guide body on a first side of the plane, the glenoid drill guide further comprising a handle extending outwardly from the guide body on the first side of the plane.

11. The glenoid drill guide of claim 10, wherein the guide body comprises first and second arm mounting interfaces, each of the first and second arm mounting interfaces being configured to releasably secure the proximal portion of the outrigger arm to a different portion of the guide body such that the distal portion of the outrigger arm extends beyond the distal end of the guide body in a different orientation relative to the first and second lumens of the guide body.

12. The glenoid drill guide of claim 11, wherein the first lumen of the guide body defines a first longitudinal axis, the second lumen of the guide body defines a second longitudinal axis, the first and second longitudinal axes collectively define a plane, the first arm mounting interface is configured to releasably secure the proximal portion of the outrigger arm to the guide body such that the distal portion of the outrigger arm extends beyond the distal end of the guide body on a first side of the plane, and the second arm mounting interface is configured to releasably secure the proximal portion of the outrigger arm to the guide body such that the distal portion of the outrigger arm extends beyond the distal end of the guide body on a second side of the plane.

13. The glenoid drill guide of claim 12, wherein a width of the guide body is greater than a width of the outrigger arm.

14. The glenoid drill guide of claim 13, wherein the implant insert comprises a coupler moveable between a first position relative to the guide body in which the implant insert is able to slide longitudinally within the first lumen of the guide body and a second position relative to the guide body in which the implant insert is prevented from sliding longitudinally within the first lumen of the guide body.

15. The glenoid drill guide of claim 14, wherein the first bone block engagement insert comprises a first compression member for releasably securing the first bone block engagement insert in the lumen of the implant insert, and the second bone block engagement insert comprises a second compression member for releasably securing the second bone block engagement insert in the second lumen of the guide body.

16. The glenoid drill guide of claim 15, further comprising:
a handle; and
first and second handle interfaces integral with the guide body, the first handle interface being configured to releasably secure the handle to the guide body such that the handle extends outwardly from the guide body in a first orientation relative to the first and second lumens of the guide body, and the second handle interface being configured to releasable secure the handle to the guide body such that the handle extends outwardly from the guide body in a second orientation relative to the first and second lumens of the guide body that differs from the first orientation.

17. The glenoid drill guide of claim 16, wherein the first lumen of the guide body defines a first longitudinal axis, the second lumen of the guide body defines a second longitudinal axis, the first and second longitudinal axes collectively define a plane, the first handle interface is configured to releasably secure the handle to the guide body such that the handle extends outwardly from the guide body on a first side of the plane, and the second handle interface is configured to releasably secure the handle to the guide body such that the handle extends outwardly from the guide body on a second side of the plane.

18. A method of repairing glenoid bone loss, the method comprising:
providing a guide body having proximal and distal ends and defining first and second lumens each extending between the proximal and distal ends;
securing an implant insert in the first lumen of the guide body such that a distal end of the implant insert is proximal to or flush with the distal end of the guide body, the implant insert defining a lumen extending between a proximal end and the distal end of the implant insert;
securing a first bone block engagement insert in the lumen of the secured implant insert such that a distal end of the first bone block engagement insert is distal to the distal end of the guide body, wherein the first bone block engagement insert defines a first lumen extending between a proximal end and the distal end of the first bone block engagement insert;
securing a second bone block engagement insert in the second lumen of the guide body such that a distal end of the second bone block engagement insert is distal to the distal end of the guide body, the second bone block engagement insert defining a second lumen extending between a proximal end and the distal end of the second bone block engagement insert;
coupling a bone block to the distal ends of the secured first and second bone block engagement inserts;
maneuvering the guide body with the coupled bone block to position the bone block against a glenoid bone;
inserting a K-wire through the first lumen of the secured first bone block engagement insert or the second lumen of the secured second bone block engagement insert while the coupled bone block is positioned against the glenoid bone to pin the bone block to the glenoid bone;
removing the first bone block engagement insert from the lumen of the secured implant insert after the K-wire is inserted;
drilling into the bone block through the lumen of the secured implant insert after the first bone block engagement insert is removed;
removing the implant insert from the first lumen of the guide body after the bone block is drilled; and
passing an implant through the first lumen of the guide body to secure the bone block to the glenoid bone after the implant insert is removed.

19. The method of claim 18, wherein inserting the K-wire through the first lumen of the secured first bone block engagement insert or the second lumen of the secured second bone block engagement insert while the coupled bone block is positioned against the glenoid bone to pin the bone block to the glenoid bone comprises inserting the K-wire through the first lumen of the secured first bone block engagement insert.

20. The method of claim 18, further comprising:
attaching a proximal portion of an outrigger arm to the guide body such that a distal portion of the outrigger arm extends beyond the distal end of the guide body; and
adjusting a position the outrigger arm relative to the guide body to engage the coupled bone block.
